(19) United States
(12) Patent Application Publication
(10) Pub. No.: US 2010/0198281 A1
(43) Pub. Date: Aug. 5, 2010

(54) METHODS FOR TREATING DISORDERS OF PERCEPTUAL INTEGRATION BY BRAIN MODULATION

(75) Inventors: C.Y. Joseph Chang, Houston, TX (US); Purvis H. Bedenbaugh, Greenville, NC (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: C.Y. Joseph Chang, MD, PA, Houston, TX (US)

(21) Appl. No.: 12/692,550

(22) Filed: Jan. 22, 2010

Related U.S. Application Data
(60) Provisional application No. 61/185,007, filed on Jun. 8, 2009.
(60) Provisional application No. 61/148,829, filed on Jan. 30, 2009.

(51) Int. Cl. A61N 1/36; A61N 2/02; A61M 37/00

(52) U.S. Cl. 607/3; 600/13; 607/45; 604/503

(57) ABSTRACT

A method for brain modulation includes modulating a predetermined site in the brain in order to treat tinnitus and other phantom perceptions, including hallucinations and the perceived compulsion to take an action. Modulation may be performed electrically, chemically, in combination, or by other techniques. The method may involve surgically implanting a medical lead at a predetermined site, and coupling the lead to an electrical signal generator or source of drugs or other chemicals. Alternatively, a predetermined site may be modulated by means of noninvasive electrical modulation such as electromagnetic induction, or other means such as by creating lesions with methods such as heating, freezing, or radiation treatment, or by virus-mediated cell destruction or functional inactivation.

### CROSS-REFERENCES TO RELATED APPLICATIONS

The present application is a non-provisional of, and claims the benefit of priority of U.S. Provisional Patent Application Nos. 61/148,829 (Attorney Docket No. 027533-000200US) filed Jan. 30, 2009, and 61/185,007 (Attorney Docket No. 027533-000110US) filed Jun. 8, 2009. The entire contents of each of the above listed Provisional Patent Applications is incorporated herein by reference.

### BACKGROUND OF THE INVENTION

1. Field of the Invention

The present disclosure relates generally to methods and apparatus for modulation of the brain. More particularly, the present disclosure relates to methods and apparatus for neuromodulation of the brain in the ventral striatum, caudate nucleus, putamen, and substantia nigra pars reticulata, in order to treat phantom perceptions and disorders of perceptual integration.

Many people perceive phantom sensations, which are sensory experiences without direct correspondence in the world outside a person's central nervous system. Such sensations may be continuous, intermittent or non-specifically modulated, but they can also be related to identifiable triggers, such as the direction of gaze for tinnitus, or nasal airflow for phantosmia. Some examples of perceived phantoms are tinnitus, pain, kinesthetic qualia of lost body parts, and hallucinated voices or images. Often these phantom perceptions relate to abnormal brain activity in a part of the brain with a characteristic sensory or cognitive function. Such abnormal activity results from factors intrinsic to the brain area, as in Lewy body diseases, or it may result from factors related to the interconnections of the brain area. Simple examples of phantom perceptions relating to a change of connectivity are pain and other perceptions related to amputation or deafferentation, or tinnitus arising from auditory injury. However it arises, the abnormal brain activity is not identical to the activity in those brain areas when they are engaged in normal physiological processes. Phantom perceptions can occur when such abnormal brain activity is integrated across brain systems.

Phantom perception requires that the abnormal brain activity be differentiated from spontaneous, ongoing or quiescent activity, and interpreted as activity that could arise in the course of normal physiological function. The interpretive process includes characterization of the percept's magnitude and other qualia, such as pitch, timbre, accent, color, shape, texture, hotness, coolness, and heaviness. The perceptions may include composites of qualia, such as identifiable faces or body parts. The interpretive process may also include an affective or other type of evaluative component. For example, the phantom may induce fear, may have a volition attributed to it, or may generate a compulsion to act upon the phantom. Some phantom perceptions may be of no particular consequence, while others may have serious consequences, or be perceived as intolerable.

Existing approaches to treating phantom sensations or hallucinations by brain modulation aim to modify brain activity in areas associated with the modality of the phantom, such as electrical modulation of the auditory cortex in treatment of tinnitus, and repetitive transcranial magnetic stimulation of the auditory cortex for the treatment of auditory verbal hallucinations. Other therapies focus on making the sensations less compelling or intrusive, such as antidepressant therapy for hallucinated voices, or tinnitus retraining therapy, which trains patients to respond to the perceived phantom sounds as part of the irrelevant acoustic background. Modulating a phantom perception to make it less compelling or noticeable can cause it to diminish or to disappear. This is because the property of being compelling or noticeable is associated with neuronal plasticity processes, which can reinforce the perception. When such neuroplasticity mechanisms disengage, the phantom can be reduced or disappear. Examples of perceptual correlates of neuronal plasticity mechanisms include compellingness as a basis for action, intrusiveness into general and executive thought processes or other tendency for the perception to become the focus of attention, and emotional associations or affect. In other words, decoupling the integration of the abnormal activity can lead to its elimination. Therefore treatment with devices, systems and methods which interfere with the integration of a phantom sensation with brain mechanisms of perception have the potential to obscure or suppress the phantom perception during the treatment; or to deliver an enduring relief from the phantom perception after transient treatments.

It would therefore be desirable to provide improved methods and apparatus for treating sensory phantoms and hallucinations that are more effective than current treatments. It would also be desirable to identify other target areas of the brain that may be treated by neuromodulation in order to more effectively reduce or eliminate perceptions of phantom sensations. Furthermore, it would also be desirable to target therapy specific to the most offensive manifestation or manifestations of the integration process, in addition to or in place of modulating than the phantom modality itself.

2. Description of Background Art

Patents disclosing neurostimulation of the brain to treat various neurological disorders include U.S. Pat. Nos. 7,346,395; 7,315,761; 6,687,525; 6,425,852; 5,716,377; 5,683,422. Other related patents include U.S. Pat. Nos. 7,033,312; 7,076,307; 7,309,348; 6,871,098; and 6,128,537. Related patent publications include U.S. Patent Publication No. 2007/0213783. Related journal articles include de la Mothe et al., Cortical connection of the auditory cortex in marmoset monkeys: core and medial belt regions, J. Comp. Neuro. 2006 May 1; 496(1):27-71; Friedland et al., Feasibility of auditory cortical stimulation for the treatment of tinnitus, Otol Neurotol. 2007 December; 28(8):1005-12; Hikosaka et al., Role of the basal ganglia in the control of purposive saccadic eye movements, Physiol. Rev. 2000 July; 80(3):953-78; Lockwood et al., The functional anatomy of gaze-evoked tinnitus and sustained lateral gaze, Neurology. 2001 Feb. 27; 56(4):472-80; Lowry et al., An Absence of Tinnitus, Otol Neurotol. July; 25(4):474-8; Nicolson et al., Persistent auditory hallucinations that are unresponsive to antipsychotic drugs, Am J. Psychiatry. 2006 July; 163(7):1153-9; Yeterian E H, Pandya D N., Corticostriatal connections of the superior temporal region in rhesus monkeys, J Comp Neurol. 1998 Sep. 28; 399(3):384-402; Leopold D, Distortion of olfactory perception: diagnosis and treatment, Chem Senses. 2002 27:611-615; and Levy L M and Henkin R I, Physiologically initiated and inhibited phantosmia: cyclic unirhinal, episodic, recurrent phantosmia revealed by brain fMRI, Comput Assist Tomogr. 2000 July-August; 24(4):501-20.

### BRIEF SUMMARY OF THE INVENTION

The present invention relates to a method for modulating the brain to treat perception of phantoms. This includes, but is not limited to degenerative, neurological or psychiatric disorders such as tinnitus, perception of phantom limbs, hallucinations and pain. More particularly the method involves modulating a predetermined site in the brain, electrically, chemically, or some combination thereof, to modulate the magnitude or other qualia of the perceived phantom. Other qualia may include pitch, timbre, accent or accentuation in any modality, color, shape, texture in any modality, odor quality, hotness, coolness, heaviness, and body position or orientation. The method may also be used to treat perception of qualia compositions, such as faces or body parts, which may be identifiable as to species, or specific to an individual. By species it is meant a generic type of object living or nonliving, or copies, twins, or clones thereof. By specific to an individual, it is meant to describe a particular instance of an object, living or nonliving. The method may also be used to treat distortions of qualia, such as aberrant shape, configuration, or color of otherwise normally perceived scenes.

In this disclosure, the term “sensation” refers to neuronal activity in parts of the brain in which such activity is normally related to sensory processes of a single modality. Sensation is distinct from integrating signals from multiple modalities, from integrating sensation with motor or other behavioral processes including memory, and from the processes of neuronal plasticity. For the particular example of the auditory system, sensation refers to neuronal activity in the cochlea, cochlear nucleus, superior olivary complex, trapezoid body nuclei, inferior colliculus, ventral division of the medial geniculate body of the thalamus, and primary auditory cortex. The term perception refers to integrative brain processes which integrate sensory activity with behavioral, memory and plasticity processes, including processes which lead to conscious awareness of a sensory signal, or the ability to base behavior upon the sensory signal without conscious awareness, as in blind sight.

In a first aspect of the present invention, a method of therapeutically treating a phantom perception in a patient comprises modulating a brain target. The brain target comprises one or more of the head, body or tail of the caudate, putamen, globus pallidus internus or externus, corona radiata, zona incerta, internal capsule, fields of Forel, thalamus, subthalamic nucleus, and the substantia nigra pars reticulata. The modulation reduces the patient's awareness of the phantom perception or transforms perception of the phantom to achieve a desired therapeutic effect.

In another aspect of the present invention, a method of therapeutically treating influence from a phantom perception on a patient comprises modulating a portion of the patient's brain and reducing the influence of the phantom perception on the patient's behavior. The portion of the brain that is modulated comprises the ventral striatum.

In still another aspect of the present invention, a method of therapeutically treating compulsion due to a phantom perception on a patient comprises modulating a portion of the patient's brain and reducing patient compulsion to perform an inappropriate or maladaptive action in response to the phantom perception. The portion of the brain that is modulated comprises the ventral striatum.

In yet another aspect of the present invention, a method of therapeutically treating intrusiveness of a phantom perception in a patient comprises modulating a portion of the patient's brain, and reducing the intrusiveness of the phantom perception into the patient's thoughts, executive processes or behaviors unrelated to the phantom. The portion of the brain that is modulated comprises the head of the caudate nucleus.

In another aspect of the present invention, a method of therapeutically treating localization of a phantom perception in a patient comprises modulating a portion of the patient's brain, and reducing localization or multisensory integration of the phantom by the patient. The portion of the brain that is modulated comprises the substantia nigra pars reticulata (SNr).

In still another aspect of the present invention, a method of therapeutically treating reaction to a phantom perception in a patient comprises modulating a portion of the patient's brain, and modifying the patient's responses to the phantom perception including gestures, for example gestures performed by the phantom, messages, for example messages communicated by the phantom verbally or nonverbally, or other sequential correlates of the phantom. The portion of the brain that is modulated comprises the putamen.

In yet another aspect of the present invention, a method of therapeutically treating identifiability of a phantom perception in a patient comprises modulating a portion of the patient's brain, and reducing or transforming sensory qualia of the phantom, or interfering with multisensory integration of the phantom. This reduces the patient's ability to assign a specific identity to the phantom. The portion of the brain that is modulated comprises the caudate nucleus.

The therapeutic effect may comprise transforming, reducing, or eliminating one or more of a) qualia related to a perceived appearance of the phantom; b) perceived touch of the phantom or its movement upon or within a perceiver's body; c) presence or content of verbal or nonverbal messages projected from the phantom; d) gestures performed by the phantom; e) emotional affect of the phantom itself, its appearance, or messages projected by the phantom; f) an emotional response to the phantom by the perceiver; g) salience of the phantom as a contributing factor to motivating behavior; h) intrusiveness of the phantom into thought or executive processes not directly related to the phantom; or i) or multisensory integration or binding of the phantom. The qualia may comprise one or more of size, color, radiance, transparency, luminosity, spatial frequency, contrast, location, odor, taste, astringency, visual or tactile texture, motion, vibration, spatial orientation, kinesthesis, heat, coldness, pain, timbre, loudness, pitch, duration, or a composite of more elemental features such as may be identifiable as a general class or species, or may be identifiable as to a specific object or individual.

The brain modulation target may be modulated noninvasively. The modulating step may comprise modulating the brain with an extracorporeal signal generator coupled thereto by electromagnetic induction. The method may also comprise repeating the modulating step in subsequent sessions in order to continue relief from, or to restore relief from the phantom perception. The method may also comprise obtaining separate images of the brain, including the modulation target, and bone or other radiodense or radiointense structures, and placing the images of the brain into a common coordinate system. Landmarks in the images may be selected and a noninvasive modulation apparatus may be oriented relative to at least one of the landmarks so that the brain target may be modulated.

The method may further comprise implanting a medical lead in communication with the brain modulation target, and coupling the medical lead to a source of a modulating signal. The source may comprise one or more of an electrical pulse generator, or a reservoir of a therapeutic agent. The therapeutic agent may comprise a pharmaceutical which interacts with one or more of a) signaling by glutamate, GABA, dopamine or serotonin; b) neurotransmitter release; c) ion channels having a normal physiological function of stabilizing or changing membrane potentials; or d) second messenger systems. The therapeutic agent may interact with genetic mechanisms which control or modulate with one or more of the above mentioned chemicals, mechanisms, or systems. The therapeutic agent may induce cells to abandon their afferent or efferent synaptic relationships.

The therapeutic agent administered via the medical lead may modulate or interact with action of one or more additional therapeutic agents. The one or more additional therapeutic agents may be administered separately from the therapeutic agent. Administration of the one or more therapeutic agents may comprise injecting the one or more additional therapeutic agents into a fluid compartment, which may comprise blood, or cerebrospinal fluid. The administration of the one or more therapeutic agents may comprise sequentially injecting the one or more additional therapeutic agents via the medical lead.

The method may further comprise changing the numbers of cells in the target zone, such as decreasing the number of cells in the target site, as in creating a lesion. Creating a lesion may further comprise a lesion to a pre-defined population of cells in the vicinity of the modulation target. Examples of such target populations are cells of the striosomes, cells of the striatal matrix, and interneurons.

The method may further comprise changing the number of cells in the target site, as in adding differentiated neuronal cells or stem cells. Such cells may be added once, as by injection, and may be given partial support by administration of a therapeutic agent, such as a neurotrophin, or an agonist of one of the tyrosine kinase signaling pathways, or a chemokine or other cytokine.

The method may further comprise obtaining one or more indicators of conditions adjacent the medical lead. The one or more indicators may comprise one or more of an electrical, a chemical or an electro-chemical stimulus-response assessment, an electrical recording, a sample of a physiological fluid which may contain chemical correlates of neuronal activity, or a biomarker of a disease process. The one or more indicators may be used as a reference value and the modulation may be applied via the medical lead. One or more indicators of the environment adjacent the medical lead may also be obtained and the indicators of the environment may be compared with the indicators used as reference values. The modulating therapy may be updated based upon the comparison. The therapy may be updated by pausing the modulation or resuming the modulation.

The phantom may comprise tinnitus or a voice. The modulating step may comprise modulation according to a predetermined schedule, or may comprise controlling the modulation by provider in a single session, or in a sequence of sessions. The modulating step may comprise controlling the modulation by the patient. The modulation may be suspended when perception of the phantom is reduced to a level acceptable to the patient. The modulation may be resumed when perception of the phantom increases to a level unacceptable to the patient. The modulating step may comprise stimulating the brain with a stimulus and the stimulus may be steered within the brain.

The modulating step may comprise bilaterally stimulating corresponding target sites in the brain or bilaterally stimulating complementary target sites in the brain. Modulating may comprise unilateral stimulation of the brain or it may comprise forming a lesion in the brain. Modulating may also comprise one of cooling, heating, or irradiating a portion of the brain.

These and other embodiments are described in further detail in the following description related to the appended drawing figures.

### BRIEF DESCRIPTION OF THE DRAWINGS

FIG. 1 diagrams the process of normal perception of a sensory stimulus.

FIG. 2 diagrams brain processes related to phantom perceptions.

FIG. 3 diagrams the details of major basal ganglia circuitry.

FIG. 4 diagrams a procedure for treating a phantom perception.

FIG. 5 diagrams a procedure for treating two facets of a phantom perception by sequential application of the method.

FIG. 6 is a continuation of the procedure diagrammed in FIG. 5.

FIG. 7 is a continuation of the procedure diagrammed in FIGS. 5-6.

FIG. 8. diagrams a procedure for using measurements obtained from the vicinity of the target site to guide treatment of a phantom perception.

FIG. 9 diagrams a placement of a medical lead within a patient.

FIGS. 10A-10B illustrate the placement of a brain stimulating lead in a patient with tinnitus, whereby electrical stimulation modulates tinnitus perception.

FIGS. 11A-11D illustrates the placement of a brain stimulating lead in a second patient with tinnitus, whereby electrical stimulation modulates tinnitus perception.

FIGS. 12A-12B illustrates modulation of tinnitus by an electrical modulating signal applied via an implanted medical lead.

### DETAILED DESCRIPTION OF THE INVENTION

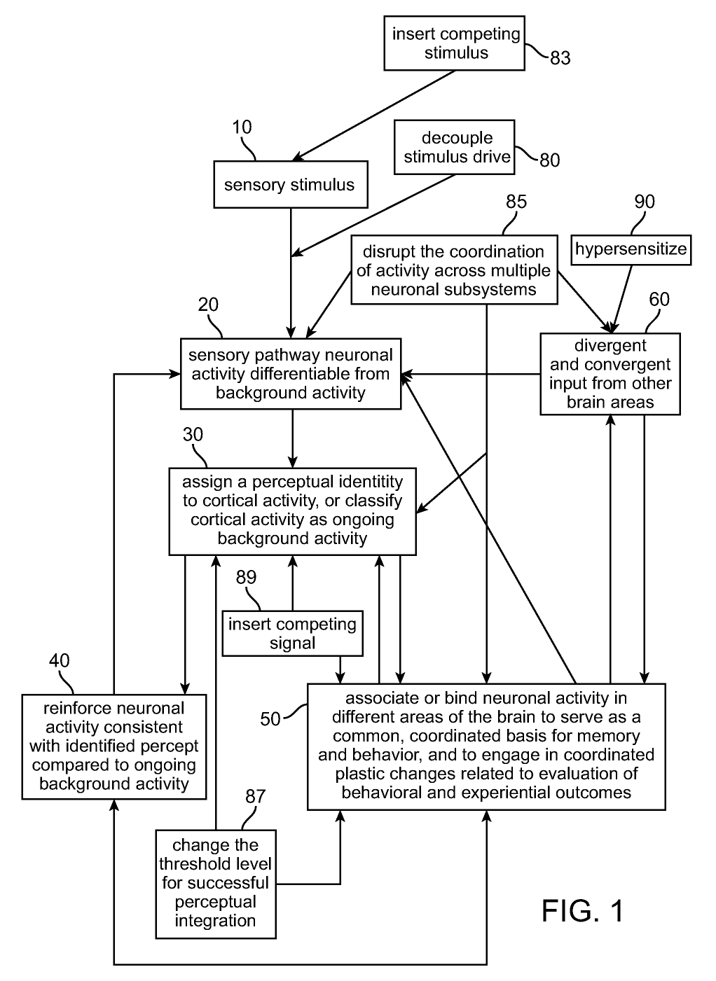

FIG. 1 describes the normal process of perception related to a sensory phantom, and processes that can influence phantom perceptions. A sensory stimulus 10 couples to identifiable neuronal activity within a sensory neuronal pathway 20. Simultaneous signals in motor pathways, brain association areas, and brain areas concerned with other modalities also contribute to sensory neuronal activity 60. This effect can be exacerbated if activity in areas outside of a specific sensory pathway 60 or its coupling to sensory brain areas 20 becomes hypersensitized 90. Such convergent and divergent connections make neuronal activity in sensory brain areas dependent upon the behavioral context. An example of a phantom perception which directly depends upon behavioral context is certain cases of tinnitus, in which depending upon the direction of gaze, the tinnitus may be perceived as present or absent. Sensory pathway activity may couple to processes 30 which identify, perceptually, the sensory neuronal activity. Alternatively, sensory pathway activity may fail to couple to such processes, as in the case of ongoing background activity. Neuronal activity with a perceptual identity is reinforced 40, for example through feedback connections and mechanisms of attention. Neuronal activity across the brain, which relates to a single perceptual identity is bound together 50, so that it is interpreted as a coordinated basis for behavior, drives coordinated plastic changes, and forms coordinated memory traces which can be retrieved together, or associatively.

Perception of a phantom sensation may be reduced or blocked by many strategies which interfere with the processes diagrammed in FIG. 1. In one example, a competing stimulus 83 can be inserted into the perceptual pathway, to compete with and obscure the phantom. Such a practice is incorporated into tinnitus retraining therapy, in which patients wear a noise generator. The noise masks the tinnitus, which makes it less compelling. A less compelling sensation recruits mechanisms of reinforcement and plasticity to a lesser degree, and so tends towards fading into the background. In another example, the stimulus drive could be decoupled by inserting a signal at a later stage of a sensory pathway 80. An embodiment of such a practice is electrically stimulating the cortex of the superior temporal plane as a treatment for tinnitus. In another example a competing signal 89 could be inserted into the processes which assign perceptual identity to sensory neuronal activity 30 or to the processes which bind neuronal activity across sensory systems. Alternatively, the threshold required to engage such processes as identification, binding and association, could be changed 87. The devices and methods disclosed herein may use such mechanisms in order to treat phantom perceptions. Directly interfering with perceptual processes such as binding can reduce the phantom, transform its form or qualia, or eliminate it entirely. Still another treatment strategy 85 is to disrupt the coordination of activity across multiple neuronal subsystems. With this strategy, individual neuronal subsystems may still generate aberrant signals, however these signals do not contribute to a common percept. The individual signals also do not change together in a coordinated way, in the manner of changes driven by bound perceptions acting through neuronal plasticity mechanisms. Without mutual reinforcement, the aberrant signals become less prominent, and may fade out of awareness.

One pathophysiological mechanism which can lead to phantom perception is decoupling stimulus drive from sensory pathways, as in tinnitus and phantom limb pain. Cellular regulatory processes in adult neurons cause them to either die, or to generate some output regardless of their input. When the input is disrupted, the generated activity does not properly represent a sensory stimulus or an organized percept. One type of phantom perception occurs when a perceptual identity is assigned to this nonrepresentative activity.

Another pathophysiological mechanism which can lead to phantom perception is to reduce the number of neurons and/or reduce their interconnections in some area of the brain, or within a network of interrelated brain areas, as in Lewy body disease. Normal brain activity has characteristic dynamics, with periods of transient or modulated activity, periods of sustained activity, periods of inactivity, such activity which may be periodic, unstructured, or otherwise aperiodic. Normally the transitions between dynamical states are well regulated. When neuronal networks are physically disrupted, the states and the transitions between them are no longer well regulated; spurious states and transitions can occur. When such spurious states are assigned a perceptual identity, they can become self reinforcing and persist for a time. A phantom perception can then occur.

Another pathophysiological mechanism which can lead to phantom perception is over activity in neuromodulatory brain circuits, or over sensitivity to their effects, as in schizophrenia other psychiatric disorders. Neuronal activity which normally would not couple to the mechanisms of perception engages the mechanisms of neuronal plasticity and becomes enhanced to the point where it is assigned a perceptual identity, and becomes bound to other neuronal activity, which itself may be spurious.

The pathophysiological mechanisms described above are exemplary, and are not intended to be an exhaustive listing. They share the aspect that an underlying neuropathology becomes problematic or turns into a disease when untoward neuronal activity is assigned a perceptual identity, and engages brain mechanisms which reinforce the pathology, persisting with or without coupling to the ambient environment.

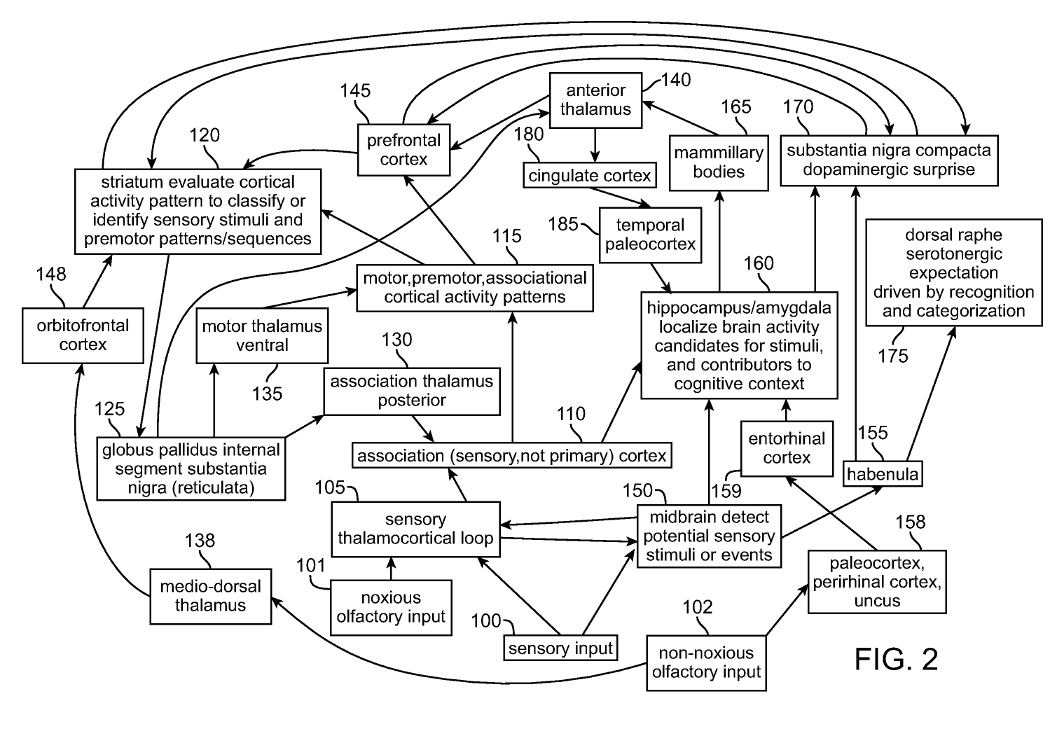

FIG. 2 expands upon this schematic, identifying brain areas which are related to specific neurological processes such as those described in FIG. 1. The arrows in this diagram represent direct influence, without specification of the sign, magnitude, or dynamical evolution of such influence. The general roles that these brain areas play are described in Table 1 below.

TABLE 1

| General Function | Brain Areas |
|---|---|
| Sensory brain activity. | 100, 101, 102, 105, 110 |
| Categorization of sensory and premotor | 115, 120, 125, 130, 135 |
| brain activity. | |
| Planning, goal directed behavior. | 120, 125, 138, 140, 145, 148 |
| Explicit contextual memory. | 110, 140, 158, 159, 160, 165, 180, 185 |
| Procedural memory and sequence | 110, 115, 120, 125, 130, 135 |
| generation, including linguistic or speech | |
| sequences. | |
| Associative clustering (binding) of brain | 110, 115, 150 |
| activity relating to a single potential | |
| rationale for present and future behavior. | |
| Evaluate behavioral outcome. | 140, 160, 165, 180, 185 |
| Direct plastic changes in synapses between | 155, 170, 175 |
| neurons whose coordinated activity relate | |
| to an evaluated behavioral outcome. | |
| Regulate stability, responsiveness, and | 165 |
| sensitivity to perturbation of neuronal | Other areas not explicitly diagrammed, but |
| circuits in general. | including the hypothalamus, reticular |
| | formation and general neuromatrix |
| | regulation of excitability. |
| | Such connections allow integration of |
| | vagal stimulation, treatment by drugs with |
| | antiepileptic or antidepressant activity in |
| | conjunction with electrical brain |
| | modulation treatment in coordination with |
| | the specific electrical and pharamacological |
| | treatments described herein. |

Each brain area identified in FIG. 2, and in Table 1, represents a potential target for modulation for treatment of phantom perception. In normal sensation and perception, all of these areas work in a coordinated fashion, and guide adaptive behavior in the present and future. In phantom perception, neuronal activity which is not properly representative of a sensory or cognitive event, or a motor plan is introduced to or is generated by one or more of these brain areas. Other neuronal circuits may (1) not effectively couple their activity to the unrepresentative brain activity, so that it has no effect upon them, (2) couple their activity to the unrepresentative activity in an uncoordinated way, or in ways that are highly dependent upon the context of ongoing activity in those brain areas, so that the unrepresentative activity introduces or appears to introduce random variability or noise to their normal proper function, or (3) respond to the unrepresentative activity as if it were representative activity of some genuine sensory event, cognitive event, or motor plan. Phantom perceptions arise in this third case. Any intervention which changes the brain response to unrepresentative activity from this third case to either of the first two cases comprises a treatment for phantom perception. From this perspective, electrical modulation of any of the areas identified in FIG. 2 is an example of a method for treating phantom perceptions.

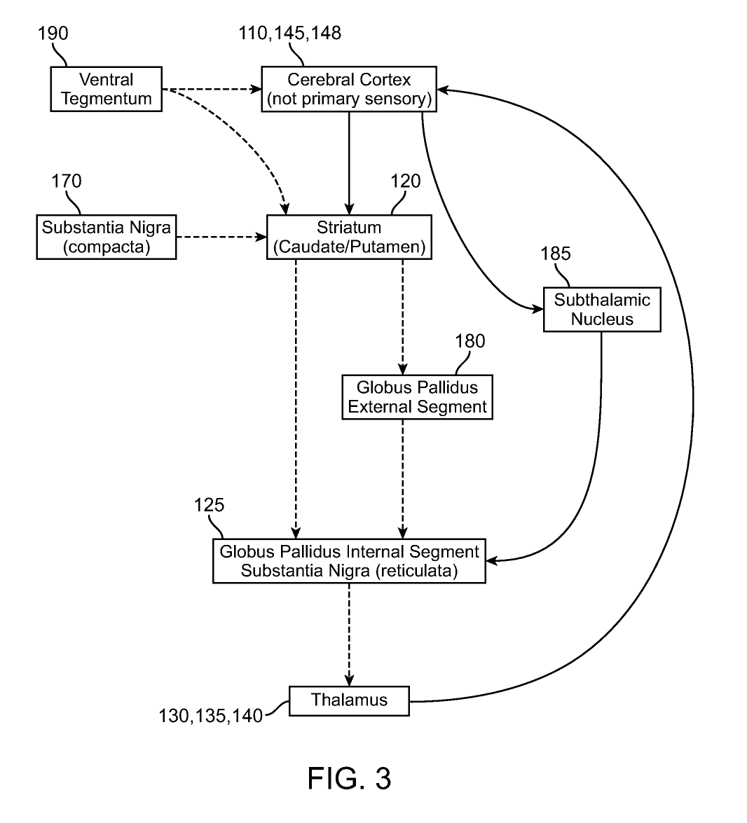

FIG. 3 diagrams the basal ganglia circuitry, which is the focus of the methods herein disclosed. Neurons in the cerebral cortex 110, 145 send an excitatory projection to the striatum 120, which is in turn composed of the caudate nucleus and putamen. The striatum sends an inhibitory projection primarily to the globus pallidus internal segment (GPi) and substantia nigra pars reticulata (SNr) 125, but also the globus pallidus external segment (GPe) 180, which in turn sends an inhibitory projection to the GPi and SNr 125. The GPi and SNr 125 send an inhibitory projection to the thalamus 130, 135, 140, and the thalamus sends an excitatory projection to the cerebral cortex 110, 145. In parallel, the cerebral cortex 110, 145 sends an excitatory projection to the subthalamic nucleus 185 which itself sends an excitatory projection to GPi and SNr 125. As previously described GPi and SNr inhibit thalamic nuclei 130, 135, 140, which excite the cerebral cortex 110, 145. Thus GPi and SNr are involved with the cerebral cortex in two parallel negative feedback loops. The striatum is modulated by dopaminergic projections from the substantia nigra pars compacta (SNc), 170 and the ventral tegmentum 190. The ventral tegmentum also modulates the cerebral cortex 110, 145 with a dopaminergic projection.

The present methods of treating phantom perceptions disrupt the engagement of perceptual processes by unrepresentative neuronal activity, or the reinforcement of the engagement of perceptual processes through the mechanisms of neuronal plasticity, or both. More particularly, the methods may interfere with the process of placing the unrepresentative activity into a category that is suitable for guiding behavior, or for generating perceptible premotor and premotor sequence activity. The methods may also reduce interference with or intrusion into normal executive processes by phantom perceptions. These goals are achieved by modulation of various portions of the brain, including, but not limited to the striatum or substantia nigra pars reticulata, electrically, chemically, or through a combination of electrically and chemically. The method may be practiced through transient or reversible means, such as through electrical stimulation through a medical lead in communication with a programmable pulse generator. The method may be practiced through means which induce an enduring effect, such as creating a lesion, or by addition of cells, such as stem cells or differentiated nerve cells, or by a gene therapy which alters the balance of inter-cellular communication by neurons involved with generation, reinforcement or perception of the phantom.

“Striatum” is a gross anatomy term for a collection of nuclei with similar neurochemical architecture that resembles a comet with its head divided by the internal capsule, and with an arcuate tail. Different domains of the cerebral cortex project to different striatal domains, so that the striatal domains become involved in particular functions. Modulation of specific striatal domains has specific effects on phantom perceptions, as will be discussed in greater detail below.

Modulating a localized domain within the caudate nucleus is not currently performed as a treatment for Parkinson's disease. Modulation treatments for Parkinson's Disease may employ modulation centered within a spatially compact brain nucleus, with the effect that the target nucleus is modulated non-specifically, and with large magnitude compared to surrounding nuclei and fiber tracts. Instead of modulating a gatekeeper nucleus, in order to have a maximal effect on a pervasive disorder, the methods disclosed herein may modulate subsystems associated with a disorder with a more targeted focus. The consequence of this approach is that a good therapeutic outcome is achieved, with limited side effects. The present methods take advantage of the peculiar interconnectivity of the striatum, in which separate functional systems rely principally upon separate domains within the striatum, while also facilitating interaction and exchange of information between the systems. In addition, the striatum is a favorable target for such treatment because the activity of neurons within the striatum has modality specificity and integrates with the limbic system in coordination with mechanisms of focused attention. In contrast, striatal neurons project to neurons with less modality specificity, without direct limbic integration. Activity in cortical neurons presynaptic to striatal neurons is more modality specific and more specific within the modality, but these neurons are more remote from the systems of focused attention and affectively driven plasticity. Thus, the striatum is an excellent target for disrupting the processes which lead to neuronal activity that is not properly representative of a sensory or premotor event which has been made accessible to perceptual systems, yet persists and/or generates a percept which encroaches upon conscious perception.

The method may be practiced by modulating the striatal tissue in the vicinity of the target in any way known to the art, whether by modulating the striatum as a whole, modulating a subset of striatal neurons which integrate with a system of neuronal plasticity such as striosomal neurons, or by altering the balance of neuronal activity within the striosomal and matrisomal striatal systems. In any application the favored method is selected on the basis of expediency and a desired balance of therapeutic effects and side effects, and such selection is made in the context of an assessment of the strengths, vulnerabilities and treatment history of individual patients. Examples of methods for modulation of a striatal target site include, but are not limited to those listed below.

A variety of lesion methods may be deployed to modulate the striatum. A lesion in the vicinity of a target probe by may be created by applying heat or cold using thermal techniques known in the art. For example, heat may be generated by an electric current passing through the tissue, such as a radiofrequency electric current, microwaves or ultrasound. Other methods of creating a lesion may include administration of a toxic or caustic agent such as alcohol, or administration of an excitotoxic agent such as glutamate. Lesions may also be created by administering radiation (e.g. gamma knife). Drug therapy may also be used such as by delivering a neurotransmitter or anesthetic agent to the target site.

A virus may be introduced in order to induce expression of a controllable agent of depolarization. Exemplary viruses include, but are not limited to adeno-associated virus (AAV) or lentivirus. The virus preferably contains a vector encoding an agent of depolarization, such as channelrhodopsin. The controllable agent may be activated by illuminating the target site, for example by placing an optical lead in communication with the target site.

Changes in the balance of activity in the striosomal and matrisomal systems of the striatum may be achieved by a number of methods such as by local injection of demorphin-saporin in the vicinity of the target site. Also, lesions may be created in the striatal interneurons by local injection of SP-PE35 at the target site. SP-PE35 is a substance P—Pseudomonas exotoxin conjugate, which selectively targets striatal cholinergic and nitric oxide synthase/somatostatinergic interneurons when injected locally into the striatum. Inducing expression of a controllable agent of depolarization may also be achieved by introducing a virus such as AAV into neurons that are related to the system of neuronal plasticity. Such a system is postsynaptic to striatal neurons. The virus is preferably suitable for retrograde transport and inducing expression of a genetic construct in striatal neurons. The construct preferably encodes an agent of neuromodulation, such as channelrhodopsin. Controllably activating the agent of modulation may be achieved by placing a medical lead at the target site, and illuminating the target site with light.

Exemplary proliferative methods may include injecting stem cells to the target site or injecting differentiated neurons to the target site. A target site may be predisposed to be supportive of injected neurons by injection of a lentivirus or AAV vector expressing a neurotrophin, tyrosine kinase, cytokine or chemokine. Stem cells or differentiated neurons may be subsequently injected to the target site. Cells expressing a controllable agent of modulation may also be injected into the target site.

Regulatable methods may include direct action upon striatal tissue. This may include electical stimulation by placing a medical lead in communication with the target site, and passing electrical current through the lead. A drug or pharmaceutical agent may also be locally administered. Examples of targets of such pharmaceutical therapy include signaling by GABA, signaling by dopamine, signaling by serotonin, ion channels related to the changes in or the stability of potentials across cell membranes, neurotransmitter release, and combinations thereof. Example drugs which may be used alone or in combination with electrical stimulation include benzodiazepines, pregabalin, gabapentin, tigabine, topimarate, ethosuximide, primidone, lamotrignine, oxcarbazepine, levetiracetam, carbamazepine, zonisamide and phenytoin. Expression of an agent of modulation may be induced by injection of a virus such as AAV or lentivirus. Such a virus preferably contains a vector encoding an agent of neuromodulation, such as channelrhodopsion or halorhodopsin. The agent of modulation may be controllably activated, for example, by placing a photonic medical lead at the target site, and illuminating the target site with a sequence of pulses of light.

Methods incorporating indirection may include inducing expression of a controllable agent of modulation in striatal neurons presysnaptic to neurons that are part of a system of neuronal plasticity. One such system includes the limbic system. This may be accomplished by introducing a virus suitable for retrograde transport, such as AAV, to the vicinity of such neurons postsynaptic to striatal neurons and the terminal field of striatal neurons. The virus preferably contains a vector for the expression of an agent of neuromodulation, for example an agent such as channelrhodopsin or halorhodopsin or a pharmacologically controllable ion channel. Placing a medical lead in communication with a target site that has been selected on the basis of a characterization of the phantom allows activation and/or modulation of the agent of modulation. For example, channelrhodopsin or halorhodopsin may be illuminated with light. A chemical compound or drug may also be released or administered in the case of a pharmacologically controllable ion channel.

Methods incorporating indirection may also include inducing expression of a controllable agent of modulation in striatal neurons postsynaptic to neurons that are part of a system related by modality to the phantom. This includes, but is not limited to the auditory association cortex in the case of tinnitus or orbitofrontal cortex in the case of phantosmia. Expression may be induced by introducing a virus suitable for anterograde transport, such as a member of the pseudorabies, rabies, or herpes families of viruses. They may be introduced to the vicinity of such neurons presynaptic to striatal neurons, and the virus preferably contains a vector for the expression of an agent of neuromodulation. For example, an agent such as channelrhodopsin or halorhodopsin or a pharmacologically controllable ion channel. A medical lead may be positioned in communication with the target site which has been selected on the basis of a characterization of the phantom. The medical lead may then be used to modulate or activate the agent of modulation, such as by illuminating the target site with light in the case of channelrhodopsin or halorhodopsin. Or, a chemical compound or drug may be released or administered in the case of a pharmacologically controllable ion channel.

Methods of incorporating indirection may also include introducing expression of a controllable agent in striatal neurons that are related both to a modality involved with a phantom perception, and also that are related to neurons that are part of a system of neuronal plasticity. Expression may be induced by introducing a virus suitable for anterograde transport to the vicinity of cortical neurons that are related by modality to the phantom. For example, a virus containing a vector suitable for inducing expression of one or more subunits of an agent of neuromodulation in striatal neurons. The agent may comprise channelrhodopsin, halorhodopsin or a pharmacologically controllable ion channel. Also, a virus may be used that is suitable for retrograde transport to the vicinity neurons that are part of a system of neuronal plasticity. The virus may also be delivered in the vicinity of a terminal field of striatal neurons, and such a virus may contain a vector suitable for inducing expression of subunits or components of an agent of neuromodulation complementary to those subunits or components introduced by anterograde transport. Thus, the neurons will express both of the agents encoded by the anterograde agent and the retrograde agent. A complete agent of neuromodulation is therefore expressed, such as halorhodopsin, channelrhodopsin or a pharmaceutically gated channel. The agent of modulation may be controlled by placing a medical lead in communication with the striatal target and illuminating the target in the case of halorhodopsin and channelrhodopsin with light. Alternatively, a drug, chemical substance, or pharmaceutical agent may be administered or a medical lead may be placed in communication with the striatal target. A pharmaceutical agent may be administered in the case of a pharmacologically controllable ion channel.

The step of modulation may be practiced by these methods, alone or in combination, or by any other method known to those skilled in the art.

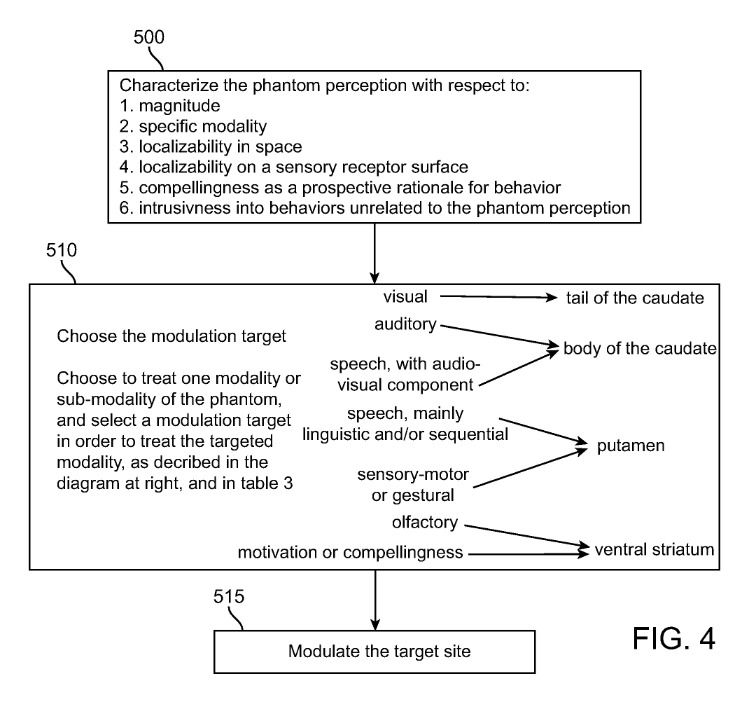

FIG. 4 diagrams a procedure for treating a phantom perception. First 500, evaluate the phantom for magnitude, compellingness, intrusiveness, localizability and specific sensory or motor modality. On the basis of this evaluation, choose a modality of the phantom, or a submodality of the phantom as a treatment objective. Select a brain target 510 that is to receive a modulation therapy and in order to treat the phantom perception in the chosen modality. The target for treating visual phantoms or gestural phantoms with a prominent visual component is the tail of the caudate. The target for treating sensory-motor or gestural phantoms not including a prominent visual component is the putamen. The target for treating auditory phantoms or speech phantoms with a prominent auditory component is the caudal portion of the head of the caudate and the body of the caudate. The target for treating motivation or compellingness is the ventral striatum. A modulation therapy is then applied to the target 515. The modulation therapy is any therapy which, applied to the target site, disrupts the integration of neuronal activity related to the phantom with perceptual systems, or the consolidation or reinforcement of neuronal activity related to the phantom through processes and systems of neuronal plasticity.

In a preferred embodiment, the modulation therapy is administered to the corresponding target sites bilaterally, treating the same structure on each side of the brain. In the preferred embodiment, two electrical modulating leads are implanted so that their modulating surfaces are in communication with the modulation targets, and its other ends are in communication with one or more implanted pulse generators and/or controllers; such systems are well known in the art. In this preferred embodiment the modulation therapy further comprises applying brief charge-balanced electrical pulses, less than 5 msec in duration, at a high rate greater than 20 pulses per second. In other embodiments, the modulation therapy may be administered to complementary sites that work together to provide a more effective result than if the sites were modulated individually. In still other embodiments, the modulation therapy may be applied unilaterally, such as when modulation is administered to treat tinnitus localized to one ear. In still other embodiments, the modulation therapy may be applied unilaterally when it provides bilateral benefit, such as when the phantom is not localized to one side of the body, or the space therearound.

As an example of the preferred embodiment, consider the application of the method diagrammed in FIG. 4 to the treatment of a phantom which is chiefly an auditory perception, such as tinnitus.

First apply FIG. 4, step 500, and characterize the phantom. The patient reports that the phantom is aversive primarily because it is enduring and unchanging in its pitch or timbre. Pitch and timbre are qualia of auditory perceptions and are normal referents of particular patterns of activity of receptor cells on the auditory receptor surface in the cochlea. The phantom does not communicate any message, and is not described as speech, music, any vocal gesture or any other sequence. The phantom is aversive secondarily because of its magnitude. The phantom is described as localized to one ear, and the patient describes location as an observable, but incidental aspect of the phantom. The phantom is quite aversive, and is a compelling basis for seeking medical treatment. It is not a compelling basis for performing any other specific behavior, save for those behaviors that may transiently modulate perception of the phantom. In every focused mental activity, the phantom intrudes as a competing focus of attention. Grave pathologies that may be related to the phantom, such as acoustic neuromas or other tumors, have been identified and treated.

In the preceding paragraph, the phantom was characterized solely on the basis of patient interviews. Additional procedures, such as structural and functional imaging, could be employed to further characterize the phantom, and are included in step 500 without limitation. For example, structural imaging could be employed in order to identify structural pathologies related to potential modulation targets and other structures with which they communicate synaptically. Functional imaging could be employed to evaluate functional correlates of the phantom, its reinforcement through engaging systems of neuronal plasticity, and its integration among sensory, perceptual, motivational and behavioral brain systems.

Based on the example characterization of the phantom 500, a modulation target is chosen 510. Because the phantom is principally auditory, the decision is made to treat the integration of an auditory phantom as a primary pathology. This maps to selecting the body of the caudate nucleus as the modulation site.

An example of modulating the target site 515 is to employ electrical stimulation by a medical lead and implantable pulse generator. A medical lead is placed on each side of the brain, so that electrical stimulating surfaces of each lead are in communication with the caudate nucleus on one side of the brain, and the body of the caudate nucleus on each side of the brain is in communication with electrical stimulating surfaces on one medical lead. The lead extension connects the medical lead to a programmable pulse generator. Electrical pulses of a predetermined duration are applied to one medical lead at a predetermined rate. The pulse magnitude is varied in steps from small up to a large magnitude. The maximum magnitude is constrained by a maximum charge density convention. At each step of electrical pulse magnitude, the magnitude of the phantom is evaluated and recorded. Pulses applied to the first lead cease, and pulses are then applied to the second lead. Pulse magnitude is controlled in the same manner as for the first lead, and the magnitude of the phantom is again evaluated and recorded. The combination of lead and stimulus magnitude yielding the most favorable outcome is employed as an ongoing electrical stimulation.

In the preceding paragraph, a predetermined rate is a relatively high rate, greater than 20 pulses per second, often 180 pulses per second. A predetermined duration is a brief duration, less than ½ of the pulse rate period. An example of a predetermined duration is 80 microseconds for the initial phase of a bi-phasic, charge-balanced pulse. The maximum current density convention varies according to engineering particulars such as the geometry of the medical lead and the stimulating surfaces, the materials and chemistry of the stimulating surfaces, and according to physiological particulars, such as the cell types and extracellular humoral environment in the vicinity of the stimulating surfaces. It is a matter of clinical judgment. A representative value for the charge density convention familiar to those skilled in the art is 65 μCoulombs/pulse phase/cm².

An alternative example of modulating the target site 515 is creating a lesion. The lesion may be created by any method known to those skilled in the art, including direct heating, heating by means of passing electrical current through the target tissue, cooling, or injection of a toxic chemical such as alcohol. Radiation may also be used (e.g. gamma knife). Other drug therapy such as a neurotransmitter or anesthetic agent may be used to modulate the target.

An alternative example of modulating the target site 515 is creating a lesion in the striosomal compartment of the striatum, which integrates with the limbic system. The limbic system can play a role in reinforcement and perpetuation of the phantom. In some instances, the involvement of the limbic system with a phantom such as tinnitus has been demonstrated with functional imaging. Functional imaging can be incorporated into the characterization of the phantom 500. If a lesion is the preferred modulation modality for a particular patient, demonstrated limbic system involvement with the phantom is a reason to prefer a lesion to the striosomal compartment to a lesion to all systems in the vicinity of the target site. A method of creating a lesion to the striosomal compartment of the striatum is to inject a molecularly targeted neurotoxin to the target site. An example of such a targeted neurotoxin is demorphin-saporin complex. Demorphin-saporin binds to mu-opioid receptors present on striosomal neurons, and is subsequently internalized and cleaved to release the ribosomal toxin saporin, thereby killing the striosomal cells.

As an example of the preferred embodiment, consider the application of the method diagrammed in FIG. 4 to the treatment of a phantom which is chiefly a chemical perception, such as phantosmia.

First apply FIG. 4, step 500, and characterize the phantom. The patient reports that the phantom is aversive primarily because it is obnoxious, and secondarily because of its frequency of occurrence and duration. The phantom is not of a noxious odor such as the odor of onions. The phantom is also aversive because of its magnitude. The phantom is quite aversive, and is a compelling basis for seeking medical treatment. It is not a compelling basis for performing any other specific behavior, save for those behaviors that my transiently modulate perception of the phantom, such as breathing, forced crying, gagging, and sticking foreign objects into the nostril. In every focused mental activity, the phantom intrudes as a competing focus of attention. Grave pathologies that may be related to the phantom, such as epilepsy or tumors, have been identified and treated.

In the preceding paragraph, the phantom was characterized solely on the basis of patient interviews. Additional procedures, such as structural and functional imaging, could be employed to further characterize the phantom, and are included in step 500 without limitation. For example, structural imaging could be employed in order to identify structural pathologies related to potential modulation targets and other structures with which they communicate synaptically. Functional imaging could be employed to evaluate functional correlates of the phantom, its reinforcement through engaging systems of neuronal plasticity, and its integration among sensory, perceptual, motivational and behavioral brain systems.

Based on the example characterization of the phantom 500, a modulation target is chosen 510. Because the phantom is principally olfactory, the decision is made to treat the integration of an olfactory phantom as a primary pathology. Olfactory perceptions integrate into conscious awareness and perception in a pathway including the olfactory bulb, paleocortex and the medial lateral orbitofrontal cortex. This maps to selecting the head and body of the caudate nucleus as the modulation site, in the zone recipient of projection from the medial lateral orbitofrontal cortex.

An example of modulating the target site 515 is to employ electrical stimulation by a medical lead and implantable pulse generator. A medical lead is placed on each side of the brain, so that electrical stimulating surfaces of each lead are in communication with the caudate nucleus on one side of the brain, and the body of the caudate nucleus on each side of the brain is in communication with electrical stimulating surfaces on one medical lead. The lead extension connects the medical lead to a programmable pulse generator. Electrical pulses of a predetermined duration are applied to one medical lead at a predetermined rate. The pulse magnitude is varied in steps from small up to a large magnitude. The maximum magnitude is constrained by a maximum charge density convention. At each step of electrical pulse magnitude, the magnitude of the phantom is evaluated and recorded. Pulses applied to the first lead cease, and pulses are then applied to the second lead. Pulse magnitude is controlled in the same manner as for the first lead, and the magnitude of the phantom is again evaluated and recorded. The combination of lead and stimulus magnitude yielding the most favorable outcome is employed as an ongoing electrical stimulation.

In the preceding paragraph, a predetermined rate is a relatively high rate, greater than 20 pulses per second, often 180 pulses per second. A predetermined duration is a brief duration, less than ½ of the pulse rate period. An example of a predetermined duration is 80 microseconds. The maximum current density convention varies according to engineering particulars such as the geometry of the medical lead and the stimulating surfaces, the materials and chemistry of the stimulating surfaces, and according to physiological particulars, such as the cell types and extracellular humoral environment in the vicinity of the stimulating surfaces. It is a matter of clinical judgment. A representative value for the charge density convention familiar to those skilled in the art is 65 μCoulombs/pulse phase/cm².

Often phantom perceptions are not well localized to a particular spatial location on or around the body, even to one side of the body. Pathways connecting the two sides of the brain are summarized in Table 2 below. In the normal circumstance, unrepresentative activity is suppressed via inter-hemispheric brain connections. This is because unrepresentative activity on one side of the brain does not couple well to a contralateral brain region that is properly functioning. Conversely, normal brain activity can engage activity in contralateral neuronal circuits, for example by entrainment, suppression or competition. If a pathology reduces brain activity, or reduces the constraints on brain activity, then neuronal activity contributing to the phantom on one side of the brain can couple to contralateral associated regions. Likewise, if the magnitude of neuronal activity contributing the phantom is large, then it may couple to contralateral associated brain regions. Therefore embodiments of treatments include modulation a striatal domain on one side of the brain, as this will often be all that is necessary. In other embodiments, a first modulation site on one side of the brain modulates the phantom perception in one functional domain, as described in Table 3. Modulation is also applied to a site contralateral to the first site, during the same session that the first site is modulated, or at a later session.

TABLE 2

| | Anatomical | |
|---|---|---|
| Commissure | Description | Physiological/Perceptual Function |
| Collicular | Connects midbrain | Bilateralizes integration of |
| commissure | nuclei. | multisensory target identity and |
| | | position information; |
| | | In cats, SNr is known to inhibit the |
| | | contralateral SC via the collicular |
| | | commissure. |
| Hippocampal | Connects dorsal | Bilateralizes contextual memory; |
| commissure | aspect of the | bilateralizes evaluation of |
| | hippocampi. | behavioral outcome |
| Corpus | Connects cerebral | Bilateralizes premotor and sensory |
| collosum | cortex bilaterally. | associative representations. |
| Posterior | Connects the | Subserves the consensual pupillary |
| commissure | oculomotor nuclei. | light reflex. |
| Anterior | Connects the | Bilateralizes olfactory processing. |
| commissure | olfactory bulbs and | |
| | temporal paleocortex. | |

TABLE 3

| | Cortical | Striatal | Effect of Striatal Modulation on |
|---|---|---|---|
| General Function | Domains | Domains | Phantom Perception |
| Motivational | Orbitofrontal, | Ventral | Modulate compellingness of |
| | anterior | striatum | phantom perception; modulate |
| | cingulate | | prosodic components or nonsteady |
| | | | modulation of phantom perceptions |
| | | | and gestures, including phantom |
| | | | voices; modulate magnitude and |
| | | | qualia of olfactory phantoms. |
| Executive | Dorsolateral | Head of | Modulate intrusiveness of phantom |
| | prefrontal, | caudate | sensations. |
| | posterior | | |
| | parietal | | |
| Categorical/Sensory | Temporal, | Body, tail | Modulate magnitude and qualia of |
| | ventrolateral | and | phantom perception. |
| | prefrontal | caudal | |
| | | head of | |
| | | the of | |
| | | caudate | |
| Motor/Sequential | Premotor, | Putamen | Modulate magnitude, perceived |
| | SMA, | | sequence and other qualia of |
| | somatosensory | | phantom gestures, including vocal |
| | | | gestures. |

For phantom perceptions which can be localized to the space upon or around one side of the body, modulation of the substantia nigra pars reticulata (SNr) can reduce the localizability of the phantom, which in turn can transform unrepresentative signals into background signals that do not compellingly influence behavior. In addition to projecting to the ipsilateral thalamus, SNr sends a GABAergic projection to the contralateral superior colliculus (SC) via the collicular commissure. Electrically modulating the SNr ipsilateral to the perceived phantom interferes with integration of the phantom in the superior colliculus, and through its efferent projections, also interferes with integration of auditory phantoms in the inferior colliculus. Interference with multisensory integration via SNr modulation is preferred over modulation of the SC directly, since SC modulation can have the unwanted effect of inducing directed focus of gaze or attention, whereas SNr modulation has the effect of modulating the stability and responsiveness of neuronal circuitry involving the SC.

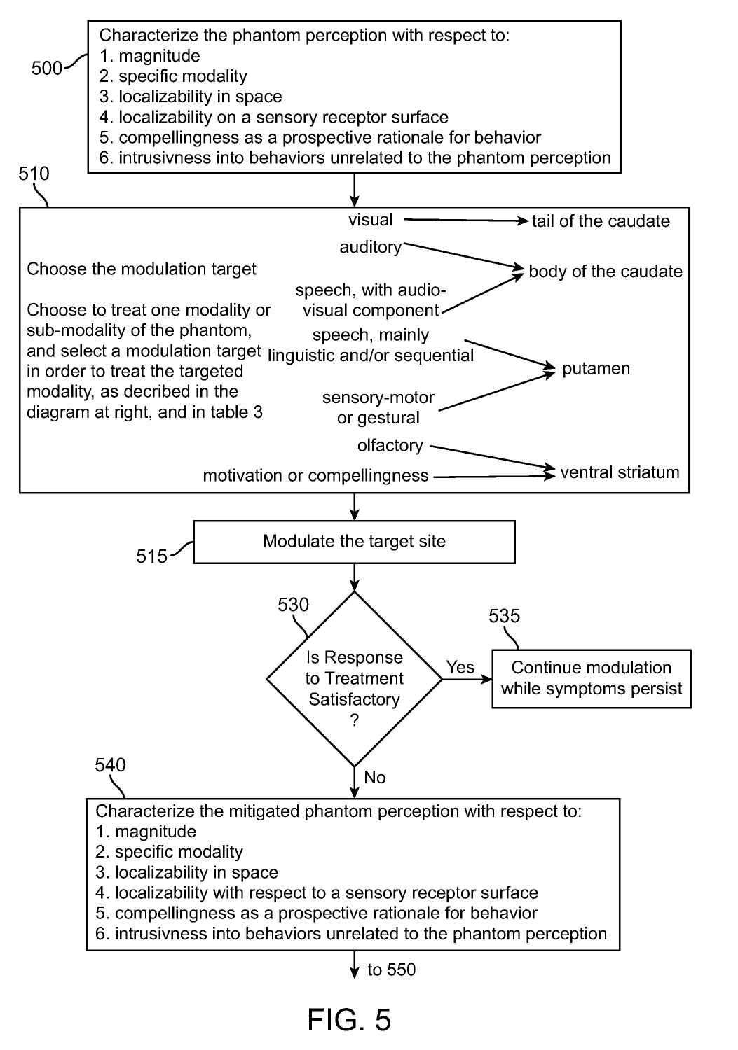

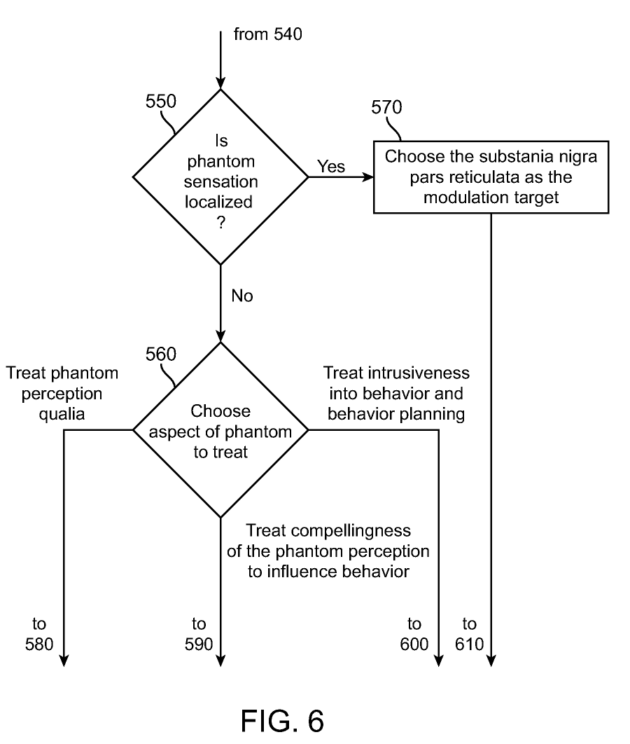

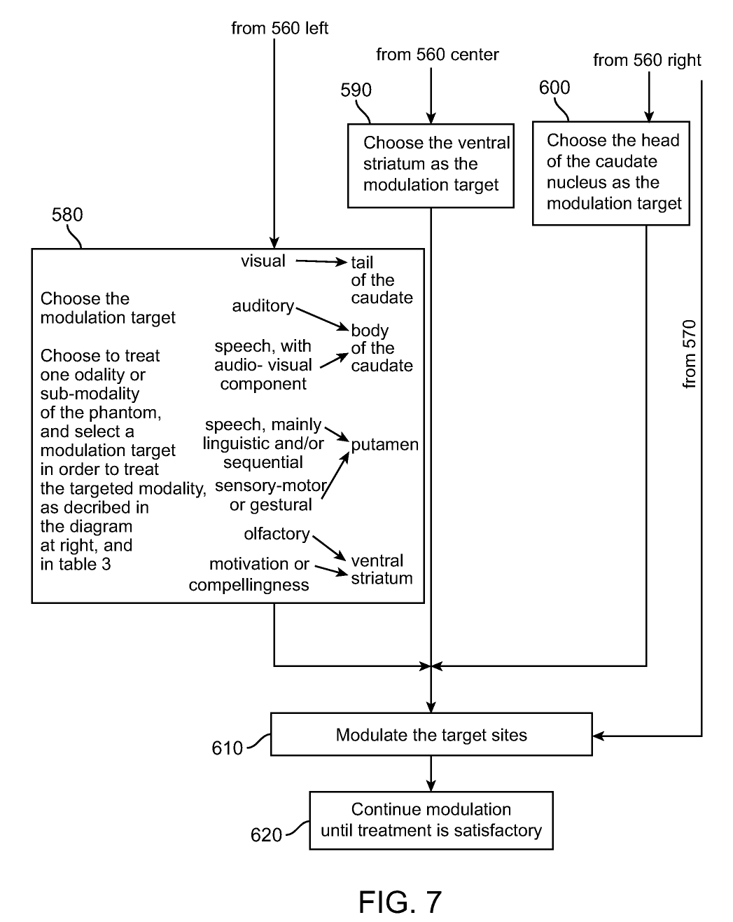

FIGS. 5, 6 and 7 diagram another embodiment of the methods disclosed herein. This embodiment illustrates how the method can be used to treat multiple aspects of a phantom, first treating a primary complaint, and continuing to treat residual pathology. First 500, evaluate the phantom for magnitude, compellingness, intrusiveness, localizability and specific sensory or motor modality. On the basis of this evaluation, a brain target is chosen 510. For phantoms which are perceived to be localized on one side of the body or one side of the space around the body, the preferred embodiment is to modulate the side of the brain contralateral to the location of the perceived phantom. The target for treating visual phantoms or gestural phantoms with a prominent visual component is the tail of the caudate. The target for treating sensory-motor or gestural phantoms not including a prominent visual component is the putamen. The target for treating auditory phantoms or speech phantoms with a prominent auditory component is the caudal portion of the head of the caudate and the body of the caudate. Modulation is applied to the target site 515. In the preferred embodiment, an electrical stimulating and monitoring lead is implanted so that its stimulating and recording surfaces are in communication with the modulation target, and its other end is in communication with an implanted pulse generator and/or controller, such systems are well known in the art. The treatment result is then evaluated to establish whether the clinical result is satisfactory 530. If electrical modulation successfully treats the phantom perception, continue to repeat modulation at the targeted site as symptoms reappear 535. Otherwise, the residual characteristics of the phantom remaining after the first treatment are evaluated with respect to magnitude modality, localizability, compellingness and intrusiveness 540. If the phantom is evaluated to be localized in space on or around the body 550, then the SNr is selected as a modulation target, so as to disrupt multisensory integration of the localizable phantom 570. Returning to step 550, if the phantom is not perceived as localized, the residual perception remaining after the initial treatment is evaluated 560 to identify which of three aspects of the phantom are deemed to be most problematic: (1) the presence of the phantom perception as an identifiable object, (2) the intrusiveness of the phantom into ongoing behavior and into planning of prospective behaviors, or (3) the compellingness of the phantom as a basis for behavior. For example, consider the common case that the presence or sound of a phantom voice is regarded to be more problematic than the desire to base prospective behavior on messages communicated by the same voice. Then the body of the caudate would be selected as a modulation target, in order to make the neuronal activity related to the perceived sound of the voice less distinguishable from normal background activity 580. Alternatively, if the intrusiveness of the voice interferes with execution of behaviors and planning of future behaviors is most problematic, then the head of the caudate is selected as the modulation target 600. Alternatively, if the tendency or desire to act upon messages communicated by the voice is most problematic, then the ventral striatum is selected as the modulation target 590. Once the new modulation target is selected, the new modulation target is electrically modulated, or both targets are electrically modulated 610, and such modulation continues while symptoms persist (620).

It will be clear to those skilled in the art that the perceived phantom need not be linked to specific sensory qualia, but may be instead an ideation. Recall that Table 3 indicates that the head of the caudate is a suitable target for brain modulation treating the intrusiveness of a phantom perception. Modulation of the head of the caudate is also a suitable treatment for intrusive ideations, such as a fear of contamination, as is common in obsessive-compulsive disorder. Such fears may be associated with remembered, imagined or fantasized qualia, but may be clearly distinguishable from an actual perception. Likewise modulation of the ventral striatum is a suitable site for treating compulsive behaviors which are not obsessive, such as compulsive eating, or stereotypic behaviors, such as are common in autism, or the compulsion to consume a substance, such as in addictions to alcohol, nicotine and other drugs.

A preferred embodiment of the treatment for phantom perceptions is to choose the primary modulation site on the basis of the modality of the phantom, and then to select a second modulation site in order to interfere with multisensory integration of the phantom, or to treat affective components of any residual phantom remaining after modulation of the primary site. For some phantoms, however, it may be preferred to choose the primary site to interfere with processes of multisensory integration, compellingness or intrusiveness. A phantom comprising an embodied voice, or a visual phantom communicating with meaningful sequential/linguistic, metaphorical or ideographic gestures, is a candidate for targeting the substantia nigra pars reticulata as the primary modulation target, with a modality related or affective component related site as a secondary modulation target.

Because of the interconnectivity between the two sides of the brain, when treating a phantom perceived to be localized to one side of the body or the space around the body, it may not be necessary to modulate the striatum contralateral to the perceived phantom location. Modulation of the striatum ipsilateral to the perceived phantom location can still provide therapeutic benefits. Contralateral modulation is the normal preferred embodiment because (1) the contralateral striatum has more direct influence on brain areas most involved with processing stimuli and events on the opposite side of the body, and (2) it keeps open approaches to the SNr on the side of the brain ipsilateral to the perceived phantom location.

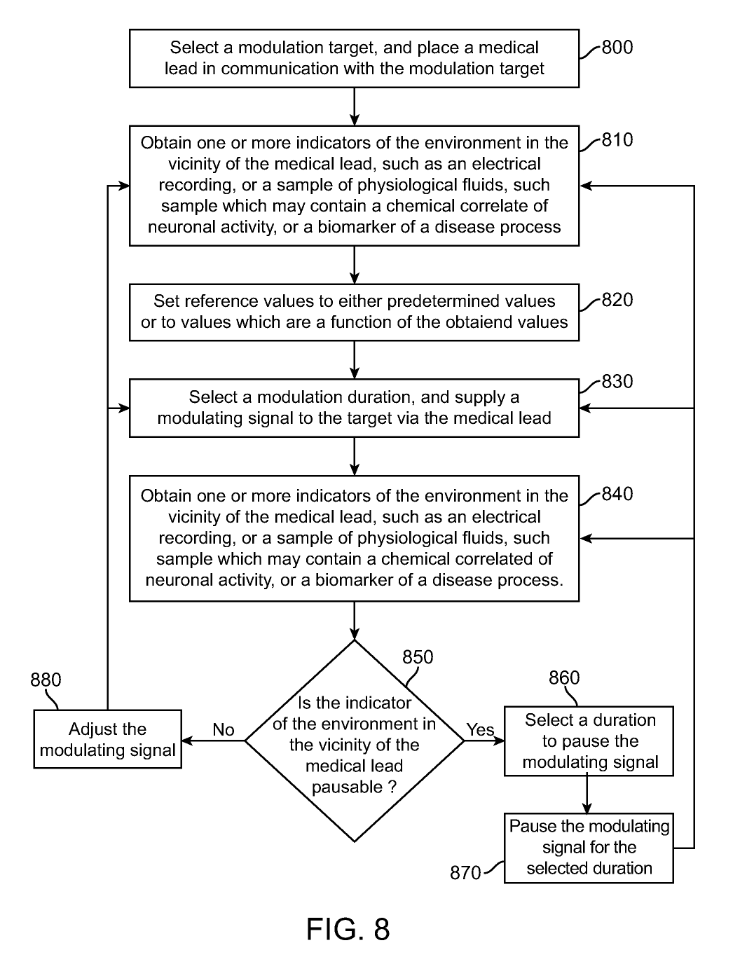

Additional embodiments of the treatment method may make use of measurements of the environment in the vicinity of a medical lead, as described in FIG. 8. Such embodiments may be enabled through an automatic algorithm, or through the intervention of a healthcare professional. An example of a medical lead which can support such measurements is described in a U.S. Patent Publication No. 2008/0027504 to Bedenbaugh, “Lead and methods for brain monitoring and modulation.” Referring to FIG. 8, a brain target for a modulation therapy is selected according to the preceding disclosure, and a medical lead, which supports obtaining signals or indicators related to the environment in its vicinity, is placed in communication with the modulation target 800. At least one such signal or indicator is obtained 810 using the medical lead, such signals and indicators may be electrical, as in the examples of EEG or field potential recordings. Such signals and indicators may be chemical, as in the example of obtaining samples of the extracellular fluid, which may contain assayable correlates of neuronal activity such as potassium ions, amino acids, catecholamines, a chemical supplied by the lead, or their metabolites or synthetic or catabolic enzymes. Such indicators may be static, as in a measure of the level or magnitude of the indicator, or they may be dynamic, as in the power spectrum of a time series of indicator values, or a stimulus-response time series. In obtaining a stimulus-response time series, the stimulus and the response may be either chemical or electrical, alone or in combination. The stimulus-response relationship may be transformed to any advantageous format known to those skilled in the art, such as a system theoretic transfer function. Reference values 820 are set for comparison to indicators which may be obtained in the future. Such reference values may be predetermined, or they may be a function of the initial measured values. Often the function is one of identity, a constant proportion. A duration is selected 830 for the initial application of the modulating signal. Such duration is often chosen for convenient application of the method. An example of a duration is the interval between follow-up visits to a healthcare provider. Other suitable durations are short, such as a few minutes to a few hours, or linked to the diurnal cycle, such as 24 hours or overnight. A modulating signal is supplied to the brain target via the medical lead. Such a signal may be chemical, electrical or a combination thereof. After the modulating signal has been applied for the selected duration, new indicators of the environment in the vicinity of the medical lead are obtained 840. Obtaining such measurements may, but need not, interrupt application of the modulating signal. The new indicators are compared to reference values 850. If the level of the signal is pausable, then a duration for pausing the modulating signal is selected 860. Examples of a pausable comparison are the case that the new indicators exceed the reference values, or the integral of the product or squared difference of a new indicator power spectrum with the reference indicator power or spectrum exceed a predetermined threshold or achieve statistical significance. The pause duration may be zero. Pause duration may be chosen to be convenient to the application of the method. An example of a convenient duration is the interval to the next follow-up visit to a healthcare professional. Other suitable durations relate to the diurnal cycle, such as 24 hours or overnight, or a constant proportion of the duration that the modulating signal was applied. The pause duration may also be chosen to achieve a favorable balance between the useful life of the source of the modulating signal and a therapeutic effect. Examples of modulating signal sources are a drug reservoir and an implantable battery. The modulating signal is paused for the selected duration 870. After the pause, indicators of the environment in the vicinity of the medical lead may again be evaluated, in a repeating cycle until the indicator is no longer pausable. Alternatively, immediately after the pause a modulation duration may be selected and modulation resumed 830. Alternatively, new indicators of the environment in the vicinity of the lead may be obtained 810. If the indicator comparison does not have a pausable outcome, the modulating signal is adjusted 880. The adjustment is often an increase in the magnitude of the modulating signal, but may be zero. After the adjustment, a modulation duration may be selected, and the modulating signal resumed for the selected duration 830. Alternatively, new indicators of the environment in the vicinity of the medical lead are obtained, and reference values are reevaluated.

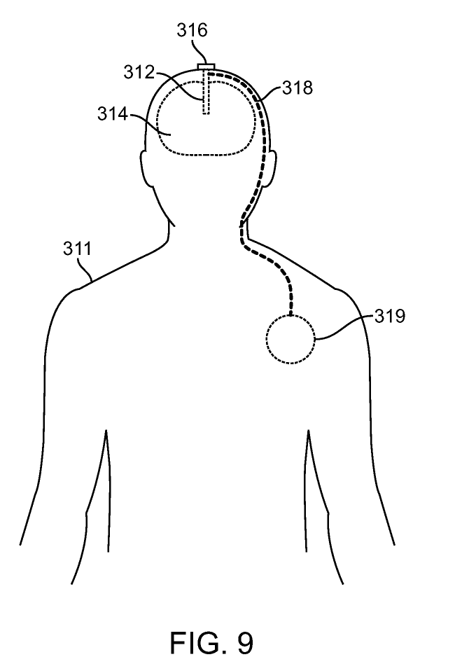

FIG. 9 illustrates implantation of a modulating lead in a patient. A craniotomy is performed in the skull of a patient 311 using surgical methods well known. A modulating lead 312, with or without brain monitoring capability, is implanted into a patient's brain 314 at the desired target, here the body of the caudate nucleus or in the vicinity thereof, and then secured to the patient's skull with an anchor fixture 316. An extension lead 318 couples the lead 312 with a stimulation source 319 such as a pulse generator and/or reservoir for a therapeutic agent. The lead 318 may run under the patient's skin or it may remain external to the patient. Similarly, the stimulation source 319 may be implanted or it too may remain external to the patient.

Electrical stimulation activates whatever neuronal signal systems are within the stimulation field. Pharmaceutical stimulation may be targeted at a subset of the neuronal signaling pathways near the site of release. Signals from the cerebral cortex couple to striatal neurons by glutamate neurotransmission, and pharmaceutical agents which interact with glutamate receptors are suitable in any of the exemplary methods disclosed herein. Other suitable targets of pharmaceutical agents interact with systemically administered drugs which modulate phantom perceptions. Examples of such targets are signaling by GABA, signaling by dopamine, signaling by serotonin, ion channels related to the changes in or the stability of potentials across cell membranes, neurotransmitter release, and combinations thereof. Example drugs which may be used alone or in combination with electrical stimulation include benzodiazepines, pregabalin, gabapentin, tigabine, topimarate, ethosuximide, primidone, lamotrignine, oxcarbazepine, levetiracetam, carbamazepine, zonisamide and phenytoin. Carbamazepine, which acts upon multiple neurochemical systems, has been reported to decrease the pitch of tinnitus in patients with both tinnitus and perfect pitch. Stimulation of the body of the caudate by a pharmaceutical agent in alternation with electrical stimulation provides relief from adaptation, and promotes enduring relief from the phantom perception in the case that continued treatment by a single modality ceases to provide relief from the phantom perception.

In a preferred embodiment, the stimulation means is a pulse generator that is optionally implantable, because such means affords the ability to target specific aspects of the phantom perception, and because it offers direct and immediate control of and access to the treatment at any time during the day or night. As is known in the art, drugs may interact with signaling by GABA, dopamine or serotonin, with ion channels related to changes in neuronal cell membrane potential, or the stability of neuronal cell membrane potential, with neurotransmitter release, or with multiple neurochemical systems. Such embodiments where the stimulation means comprises a drug are advantageous because they require implantation of less metal in the brain, thereby increasing compatibility of the implant with magnetic resonance imaging. In still other embodiments, the body of the caudate may be stimulated by noninvasive means, such as electromagnetic induction including transcranial magnetic stimulation (TMS). An example of such noninvasive means for delivering electrical stimulation by an extracorporeal device coupled to a neurostimulation target by means of electromagnetic induction is described in U.S. Pat. No. 6,425,852 to Epstein et al. In still other embodiments, electrical and chemical stimulation could be combined, without violating the spirit of this invention.

The following exemplary embodiments of methods and apparatus will be described in the context of modulation of the brain as a treatment for tinnitus. This is intended to be for illustrative purposes only and one of ordinary skill in the art will recognize that the apparatus and methods disclosed herein may be used in a number of other applications and therefore are not limited to treatment of tinnitus.

A reported medical incident which demonstrates empirically that the striatum and/or neighboring structures can have a controlling effect on the perception of phantom sensations is described by Lowry et al. (2004). An otolaryngologist experienced constant tinnitus for over 40 years. The perception stopped after he suffered a stroke. The report describes the qualia of the phantom perception: “The tinnitus could be described as always present and usually as a high-pitched pure tone. Usually it was left in the left ear.” An enduring total relief from the phantom perception of tinnitus ensued after the stroke. The radiologist described the appearance of the lesion in a magnetic resonance imaging study as acute left corona radiata CVA (cerebrovascular accident, also known as stroke) along the middle cerebral artery distribution. The CVA was located in the left hemisphere and involved the white matter in the more dorsal part of the corona radiata of the frontoparietal cortical area. In addition, there was involvement of the striatum, including the body of the caudate and the caudodorsal aspect of the putamen. The lesion included the white matter at the junction between these two neostriatal structures. As such, it most likely involved thalamocortical radiations and corticothalamic projections, in addition to corticocortical fibers running in the superior longitudinal fasciculus.

A CVA ipsilateral to the usual perceived location of the phantom was sufficient to eliminate the phantom perception. None of the structures involved are a part of either the lemniscal or nonlemniscal auditory pathways familiar to those skilled in the art, or known centers of higher order auditory integration, such as the cortical structures of the superior temporal plane and their interconnections. Hearing tests (audiograms) before and after the CVA showed no change in auditory acuity. This case report illustrates that the CVA, not obviously related in any way to sensory processes related to the perceived sensation, interfered with perceptual processes, and that interference with such processes is a sufficient means for treating the phantom perception of tinnitus. It is clear to those skilled in the art that the lesion simultaneously compromised a portion of the striatum along with adjacent gray and white matter structures, including thalamocortical fibers, ispsilateral to the tinnitus. From the experience with CVA, it is not obvious which structure, or combination of structures are the necessary or sufficient targets for the practice of electrical stimulation for the relief of tinnitus. However, electrical stimulation of the body of the caudate nucleus now appears to be sufficient treatment for relief of tinnitus. Because the caudate body is not a part of the auditory pathway, hearing acuity is not affected. It is also clear to those skilled in the art that tinnitus is a particular example of the more general syndrome of phantom auditory perception.

Electrical stimulation of the body of the caudate nucleus has been performed in two patients, with longstanding tinnitus, intraoperatively during placement of a stimulating lead for the treatment of Parkinson's disease. One patient had sustained relief from tinnitus postoperatively, while the other patient had relief only during electrical stimulation of the body of the caudate nucleus.

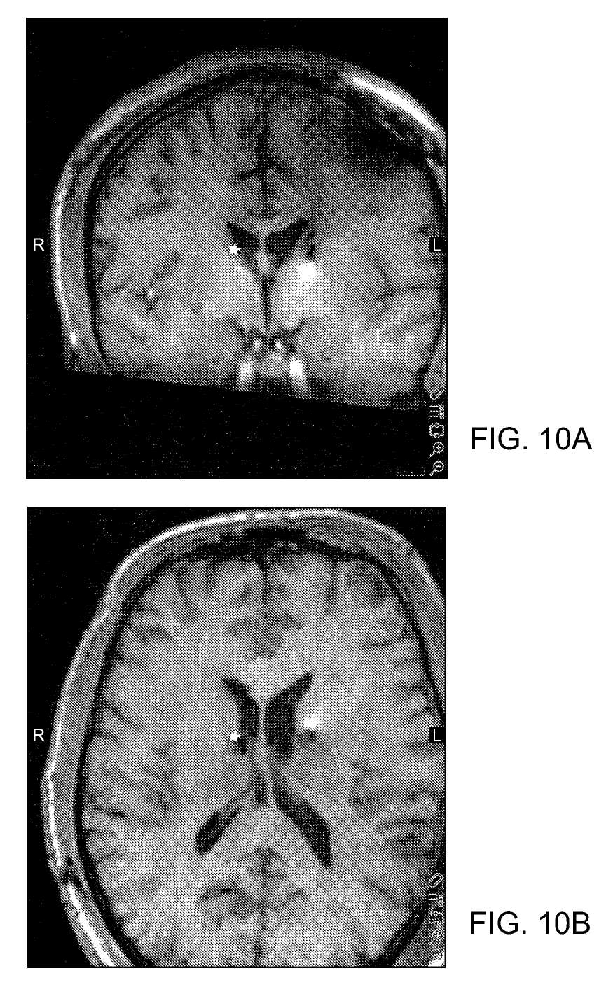

The first patient is a 51 year old male, with 10 to 20 dB bilateral sensorineural hearing loss across all frequencies, and tinnitus for 13 years. The tinnitus quality was described as broadband or “noiselike.” The patient rated the tinnitus as 5 in both ears, on an ordinal rating scale for perception magnitude ranging from 0 to 10, with 0 denoting no tinnitus, and 10 denoting the loudest possible tinnitus. The tinnitus was not localizable. The patient reported that medications for Parkinson's disease made the tinnitus louder. During image-guided surgery to implant a Medtronic 3387 electrical stimulation lead, stimulation was applied to points along a trajectory passing through the body of the caudate nucleus and terminating in the right subthalamic nucleus. Electrical stimulation located dorsal to the body of the caudate had no effect upon the perception of tinnitus. Electrical stimulation within the body of the caudate nucleus reduced the tinnitus magnitude to a level of 1 in both ears. In FIGS. 10A-10B, postoperative magnetic resonance (MR) images after bilateral subthalamic nucleus implantation for Parkinson's disease, the stimulation site is marked by a white star in coronal (FIG. 10A) and axial (FIG. 10B) planes. Ring 3 (most proximal) of the stimulation lead was the positive electrode, and rings 0 (most distal), 1, and 2 were common negative electrodes. Biphasic pulses 90 microsec in duration of the initial phase were presented at 185 pulses per second. Stimulus duration was 30 seconds. Stimulus level ramped from 3 to 8 volts over ten seconds, and then rapidly back down to 0 volts. The patient reported tinnitus suppression during the ascending phase, beginning at 5 volts. The patient and surgeon communicated normally using vocal speech while the electrical stimulation was ongoing. On the following postoperative day the patient rated the tinnitus magnitude as 0 in both ears, and it continued at zero for a period of 2 months.

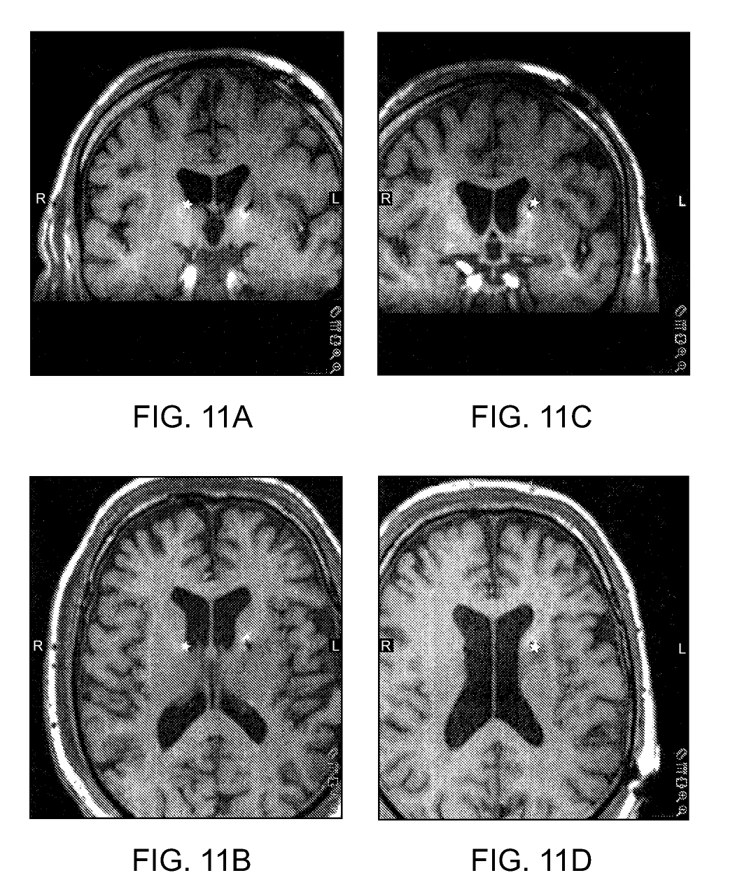

The second patient is a 67 year old male with severe bilateral sensorineural hearing loss above 2 kHz and tinnitus for 45 years. The tinnitus quality was described as constant, musical, and tonal. The patient rated tinnitus magnitude as 4 in both ears, on the rating scale previously described above. The tinnitus was not localizable. During image-guided surgery to implant Medtronic 3389 electrical stimulation leads on both sides of the brain, stimulation was applied to a point along each trajectory passing through the body of the caudate and terminating in the subthalamic nucleus. In FIGS. 11A-11D, postoperative MR images that were obtained after bilateral subthalamic nucleus implantation for Parkinson's disease, the stimulation site is marked by a white star (FIG. 11A: coronal plane, right; FIG. 11B: axial plane, right; FIG. 11C: coronal plane, left; and FIG. 11D: axial plane, left). Stimulating electrode polarity was the same as in the first patient. Bi-phasic pulses 60 microsec in duration in the initial phase were presented at 180 pulses per second. Stimulus duration was 90 to 120 seconds at each voltage step. Stimulation began at 0 volts, and was increased stepwise up to 8 volts. The body of the right caudate nucleus was stimulated first. The patient and surgeon communicated normally using vocal speech while the electrical stimulation was ongoing. Hearing tests before and after sequential bilateral caudate body stimulation showed no change in hearing acuity.

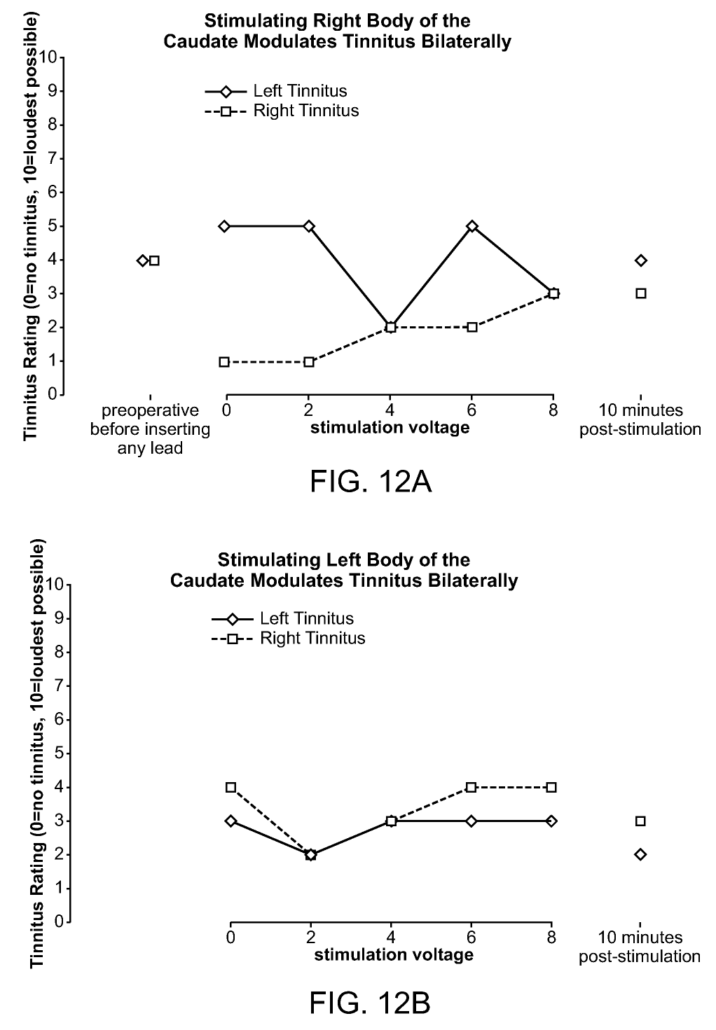

Stimulating each side of the body of the caudate modulated the perceived tinnitus on both sides, as shown in FIGS. 12A-12B. Stimulus voltage is plotted on the horizontal axis, and tinnitus magnitude in each ear is plotted on the vertical axis. In each case, there is an identifiable stimulus magnitude (FIG. 12A, upper panel, right side stimulation—4 volts; FIG. 12B, lower panel, left side stimulation—2 volts) that yields substantial relief to both ears simultaneously. The disparity in tinnitus perception magnitude between the two ears immediately after the lead was positioned in communication with the right body of the caudate nucleus represents a transient physiological effect known to those skilled in the art. Relief from tinnitus continued 10 to 15 minutes after stimulation was discontinued. In addition to changes in tinnitus magnitude, other qualia of the tinnitus were modulated. Right side stimulation at magnitudes of 2, 4 and 6 volts, resulted in a rapid (0.5 sec) increase of tinnitus pitch, followed by a relative decrease to the original pitch. Left side stimulation also produced change to both tinnitus magnitude and qualia. Stimulation at 4 volts resulted in a sustained increase in tinnitus pitch. Stimulation at 6 volts returned the tinnitus pitch to its original quality. Stimulation at 8 volts resulted in a transient increase in tinnitus pitch lasting about 1 sec, followed by a rapid return to the original. On the following postoperative day the patient rated the tinnitus magnitude as 3 in the right ear, and 2 in the left ear, with no change in tinnitus pitch.

The two exemplary reductions to practice of neurostimulation as a treatment for tinnitus illustrate such relief can be obtained by stimulating the body of the caudate nucleus on either side of the brain. In cases where a phantom auditory sensation is perceived as localized to one ear, a preferred embodiment of this invention is to stimulate the body of the caudate nucleus contralateral to the perceived sensation. This embodiment is preferred because the one side of the forebrain generally relates to signals originating from the contralateral side of the body. An alternative embodiment is to stimulate the body of the caudate nucleus ipsilateral to the perceived perception. Indeed, in the clinical case report, a CVA ipsilateral to the perceived tinnitus was sufficient to engender enduring total relief of tinnitus. Thus, the body of the caudate nucleus on either side of the brain may be stimulated in order to obtain relief from a phantom auditory perception that is bilateral, or localized to either side of the brain. In other embodiments, both the contralateral and the ipsilateral sides may be stimulated together.

Additionally, the exemplary data presented herein used commercially available medical leads. Other leads have also been proposed that have multiple electrodes with each electrode having several stimulation points. Those leads are advantageous in that they permit the stimulation to be directed or steered to within the target tissue. Directing the stimulation is desirable since it permits the stimulation to be delivered more accurately to a desired target site thereby achieving better clinical results as well as potentially minimizing reduced effectiveness of repeated stimulation due to brain plasticity. Moreover, the stimulation may be directed away from certain tissues in order to avoid undesirable side effects. These leads may also be combined with the delivery of a therapeutic agent to provide multiple sources of stimulation. One example of a medical lead that permits steering of the stimulation is disclosed in U.S. Patent Publication No. 2008/0027504 to Bedenbaugh. Thus, stimulation provided to the body of the caudate nucleus or other modulation targets may also be steered or directed for even better results.

In some embodiments disclosed herein, stimulation may be provided in an initial session, with subsequent follow-on sessions if needed. One of the examples disclosed above demonstrated sustained relief from tinnitus postoperatively. This suggests that stimulation can be applied in an initial session, and then reapplied as needed to restore relief from perception of a phantom auditory sensation. Such practice is particularly suited to the use of noninvasive means for brain stimulation, such as TMS.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A method of therapeutically treating a phantom perception in a patient, said method comprising:
modulating a brain target, the brain target comprising one or more of the head, body or tail of the caudate, putamen, globus pallidus internus or externus, corona radiata, zona incerta, internal capsule, fields of Forel, thalamus, subthalamic nucleus, and the substantia nigra pars reticulata; and
reducing the patient's awareness of the phantom perception or transforming perception of the phantom to achieve a desired therapeutic effect.

2. The method of claim 1, wherein the therapeutic effect comprises transforming, reducing or eliminating one or more of:
(a) qualia related to a perceived appearance of the phantom;
(b) perceived touch of the phantom or its movement upon or within a perceiver's body;
(c) presence or content of verbal or nonverbal messages projected from the phantom;
(d) gestures performed by the phantom;
(e) emotional affect of the phantom itself, its appearance, or messages projected by the phantom;
(f) an emotional response to the phantom by the perceiver;
(g) salience of the phantom as a contributing factor to motivating behavior;
(h) intrusiveness of the phantom into thought or executive processes not directly related to the phantom; or
(i) multisensory integration or binding of the phantom.

3. The method of claim 2, wherein the qualia comprise one or more of size, color, radiance, transparency, luminosity, spatial frequency, contrast, location, odor, taste, astringency, visual or tactile texture, motion, vibration, spatial orientation, kinesthesis, heat, coldness, pain, timbre, loudness, pitch, duration, or a composite of more elemental features such as may be identifiable as a general class or species, or may be identifiable as to a specific object or individual.

4. The method of claim 1, wherein the brain modulation target is modulated noninvasively.

5. The method of claim 4, wherein the modulating step comprises modulating the brain with an extracorporeal signal generator coupled thereto by electromagnetic induction.

6. The method of claim 1, further comprising repeating the modulating step in subsequent sessions in order to continue relief from, or to restore relief from the phantom perception.

7. The method of claim 1, further comprising:
obtaining separate images of the brain, including the modulation target, and bone or other radiodense or radiointense structures;
placing the images of the brain into a common coordinate system;
selecting landmarks in the images;
orienting a noninvasive modulation apparatus relative to at least one of the landmarks; and
modulating the brain target.

8. The method of claim 1, further comprising:
implanting a medical lead in communication with the brain modulation target;
coupling the medical lead to a source of a modulating signal, wherein the source comprises one or more of an electrical pulse generator, or a reservoir of a therapeutic agent.

9. The method of claim 8, wherein the therapeutic agent comprises a pharmaceutical which interacts with one or more of:
signaling by glutamate, GABA, dopamine or serotonin;
neurotransmitter release;
ion channels having a normal physiological function of stabilizing or changing membrane potentials; or
second messenger systems.

10. The method of claim 8, wherein the therapeutic agent interacts with genetic mechanisms controlling or modulating with one or more of:
signaling by glutamate, GABA, dopamine or serotonin;
neurotransmitter release;
ion channels having a normal physiological function of stabilizing or changing membrane potentials; or
second messenger systems.

11. The method of claim 8, wherein the therapeutic agent administered via the medical lead modulates or interacts with action of one or more additional therapeutic agents.

12. The method of claim 11, wherein the one or more additional therapeutic agents are administered separately from the therapeutic agent.

13. The method of claim 12, wherein administration of the one or more therapeutic agents comprises injecting the one or more additional therapeutic agents into a fluid compartment, the fluid compartment comprising blood, or cerebrospinal fluid.

14. The method of claim 12, wherein administration of the one or more therapeutic agents comprises sequentially injecting the one or more additional therapeutic agents via the medical lead.

15. The method of claim 8, further comprising obtaining one or more indicators of conditions adjacent the medical lead, the one or more indicators comprising one or more of an electrical, a chemical or an electro-chemical stimulus-response assessment, an electrical recording, a sample of a physiological fluid which may contain chemical correlates of neuronal activity, or a biomarker of a disease process;
using the one or more indicators as a reference value;
applying the modulation via the medical lead;
obtaining one or more indicators of the environment adjacent the medical lead;
comparing the one or more indicators of the environment with the one or more indicators used as reference values; and
updating the modulating therapy.

16. The method of claim 15, wherein the updating comprises pausing the modulating or resuming the modulating.

17. The method of claim 1, wherein the phantom perception comprises tinnitus.

18. The method of claim 1, wherein the phantom perception comprises a voice.

19. The method of claim 1, wherein the modulating step comprises controlling the modulation by the patient.

20. The method of claim 1, further comprising suspending the modulating when perception of the phantom is reduced to a level acceptable to the patient.

21. The method of claim 20, further comprising resuming the modulating when perception of the phantom increases to a level unacceptable to the patient.

22. The method of claim 1, wherein the modulating step comprises stimulating the brain target with a stimulus, the method further comprising steering the stimulus within the brain target.

23. The method of claim 1, wherein the modulating step comprises bilaterally stimulating corresponding target sites in the brain.

24. The method of claim 23, wherein the modulating step comprises bilaterally stimulating complementary target sites in the brain.

25. The method of claim 1, wherein the modulating step comprises unilateral stimulation of the brain.

26. The method of claim 1, wherein the modulating step comprises forming a lesion in the brain.

27. The method of claim 1, wherein the modulating step comprises one of cooling, heating, or irradiating a portion of the brain.

28. A method of therapeutically treating influence from a phantom perception on a patient, said method comprising:
modulating a portion of the patient's brain, the portion comprising the ventral striatum; and
reducing influence of the phantom perception on the patient's behavior.

29. The method of claim 28, wherein the portion of the patient's brain is modulated noninvasively.

30. The method of claim 28, further comprising repeating the modulating step in subsequent sessions in order to continue relief from, or to restore relief from the influence of the phantom perception upon the patient's behavior.

31. The method of claim 28, further comprising:
obtaining separate images of the brain, including the ventral striatum, and bone or other radiodense or radiointense structures;
placing the images of the brain into a common coordinate system;
selecting landmarks in the images;
orienting a noninvasive modulation apparatus relative to one or more of the landmarks; and
modulating the ventral striatum.

32. The method of claim 28, further comprising:
implanting a medical lead in communication with the portion of the patient's brain;
coupling the medical lead to a source of a modulating signal, wherein the source comprises one or more of an electrical pulse generator, or a reservoir of a therapeutic agent.

33. The method of claim 32, wherein the therapeutic agent is a pharmaceutical which interacts with one or more of:
signaling by glutamate, GABA, dopamine or serotonin;
neurotransmitter release;
ion channels having a normal physiological function of stabilizing or changing membrane potentials; or
second messenger systems.

34. The method of claim 32, wherein the therapeutic agent interacts with genetic mechanisms controlling or modulating with one or more of:
signaling by glutamate, GABA, dopamine or serotonin;
neurotransmitter release;
ion channels having a normal physiological function of stabilizing or changing membrane potentials; or
second messenger systems.

35. The method of claim 32, wherein the therapeutic agent administered via the medical lead modulates or otherwise interacts with one or more additional therapeutic agents.

36. The method of claim 35, wherein the one or more additional therapeutic agents are administered separately from the therapeutic agent.

37. The method of claim 36, wherein administration of the one or more therapeutic agents comprises injecting the one or more additional therapeutic agents into a fluid compartment, the fluid compartment comprising blood, or cerebrospinal fluid.

38. The method of claim 36, wherein administration of the one or more therapeutic agents comprises sequentially injecting the one or more additional therapeutic agents via the medical lead.

39. The method of claim 32, further comprising obtaining one or more indicators of conditions adjacent the medical lead, the one or more indicators comprising one or more of an electrical, a chemical or an electro-chemical stimulus-response assessment, an electrical recording, a sample of a physiological fluid which may contain chemical correlates of neuronal activity, or a biomarker of a disease process;
using the one or more indicators as a reference value;
applying the modulation via the medical lead;
obtaining one or more indicators of the environment adjacent the medical lead;
comparing the one or more indicators of the environment with the one or more indicators used as reference values; and
updating the modulating therapy.

40. The method of claim 39, wherein the updating comprises pausing the modulating or resuming the modulating.

41. The method of claim 28, wherein the phantom perception comprises tinnitus.

42. The method of claim 28, wherein the phantom perception comprises a voice.

43. The method of claim 28, wherein the modulating step comprises controlling the modulation by the patient.

44. The method of claim 28, further comprising suspending the modulating when perception of the phantom is reduced to a level acceptable to the patient.

45. The method of claim 44, further comprising resuming the modulating when perception of the phantom increases to a level unacceptable to the patient.

46. The method of claim 28, wherein the modulating step comprises stimulating the portion of the brain with a stimulus, the method further comprising steering the stimulus within the portion of the brain.

47. The method of claim 28, wherein the modulating step comprises bilaterally stimulating corresponding target sites in the brain.

48. The method of claim 28, wherein the modulating step comprises bilaterally stimulating complementary target sites in the brain.

49. The method of claim 28, wherein the modulating step comprises unilateral stimulation of the brain.

50. The method of claim 28, wherein the modulating step comprises forming a lesion in the brain.

51. The method of claim 28, wherein the modulating step comprises one of cooling, heating, or irradiating a portion of the brain.

52. A method of therapeutically treating compulsion due to a phantom perception on a patient, said method comprising:
modulating a portion of the patient's brain, the portion comprising the ventral striatum; and
reducing patient compulsion to perform an inappropriate or maladaptive action in response to the phantom perception.

53. The method of claim 52, wherein the portion of the patient's brain is modulated noninvasively.

54. The method of claim 52, further comprising repeating the modulation step in subsequent sessions in order to continue relief from, or to restore relief from the compulsion.

55. The method of claim 52, further comprising:
obtaining separate images of the brain, including the ventral striatum, and bone or other radiodense or radiointense structures;
placing the images of the brain into a common coordinate system;
selecting landmarks in the images;
orienting a noninvasive modulation apparatus relative to at least one of the landmarks; and
modulating the ventral striatum.

56. The method of claim 53, further comprising:
implanting a medical lead in communication with the portion of the patient's brain;
coupling the medical lead to a source of a modulating signal, wherein the source comprises one or more of an electrical pulse generator, or a reservoir of a therapeutic agent.

57. The method of claim 56, wherein the therapeutic agent is a pharmaceutical which interacts with one or more of:
signaling by glutamate, GABA, dopamine or serotonin;
neurotransmitter release;
ion channels having a normal physiological function of stabilizing or changing membrane potentials; or
second messenger systems.

58. The method of claim 56, wherein the therapeutic agent interacts with genetic mechanisms controlling or modulating with one or more of:
signaling by glutamate, GABA, dopamine or serotonin;
neurotransmitter release;
ion channels having a normal physiological function of stabilizing or changing membrane potentials; or
second messenger systems.

59. The method of claim 56, wherein the therapeutic agent administered via the medical lead modulates or otherwise interacts with one or more additional therapeutic agents.

60. The method of claim 59, wherein the one or more additional therapeutic agents are administered separately from the therapeutic agent.

61. The method of claim 60, wherein administration of the one or more therapeutic agents comprises injecting the one or more additional therapeutic agents into a fluid compartment, the fluid compartment comprising blood, or cerebrospinal fluid.

62. The method of claim 60, wherein administration of the one or more therapeutic agents comprises sequentially injecting the one or more additional therapeutic agents via the medical lead.

63. The method of claim 56, further comprising obtaining one or more indicators of conditions adjacent the medical lead, the one or more indicators comprising one or more of an electrical, a chemical or an electro-chemical stimulus-response assessment, an electrical recording, a sample of a physiological fluid which may contain chemical correlates of neuronal activity, or a biomarker of a disease process;
using the one or more indicators as a reference value;
applying the modulation via the medical lead;
obtaining one or more indicators of the environment adjacent the medical lead;
comparing the one or more indicators of the environment with the one or more indicators used as reference values; and
updating the modulating therapy.

64. The method of claim 63, wherein the updating comprises pausing the modulating or resuming the modulating.

65. The method of claim 52, wherein the phantom perception comprises tinnitus.

66. The method of claim 52, wherein the phantom perception comprises a voice.

67. The method of claim 52, wherein the modulating step comprises controlling the modulation by the patient.

68. The method of claim 52, further comprising suspending the modulating when perception of the phantom is reduced to a level acceptable to the patient.

69. The method of claim 68, further comprising resuming the modulating when perception of the phantom increases to a level unacceptable to the patient.

70. The method of claim 52, wherein the modulating step comprises stimulating the portion of the brain with a stimulus, the method further comprising steering the stimulus within the portion of the brain.

71. The method of claim 52, wherein the modulating step comprises bilaterally stimulating corresponding target sites in the brain.

72. The method of claim 52, wherein the modulating step comprises bilaterally stimulating complementary target sites in the brain.

73. The method of claim 52, wherein the modulating step comprises unilateral stimulation of the brain.

74. The method of claim 52, wherein the modulating step comprises forming a lesion in the brain.

75. The method of claim 52, wherein the modulating step comprises one of cooling, heating, or irradiating a portion of the brain.

76. A method of therapeutically treating intrusiveness of a phantom perception in a patient, said method comprising:
modulating a portion of the patient's brain, the portion comprising the head of the caudate nucleus; and
reducing the intrusiveness of the phantom perception into the patient's thoughts, executive processes or behaviors unrelated to the phantom.

77. The method of claim 76, wherein the portion of the patient's brain is modulated noninvasively.

78. The method of claim 77, further comprising repeating the modulation in subsequent sessions in order to continue relief from, or to restore relief from the intrusiveness of the phantom perception on the patient.

79. The method of claim 76, further comprising:
obtaining separate images of the brain, including the head of the caudate nucleus, and bone or other radiodense or radiointense structures;
placing the images of the brain into a common coordinate system;
selecting landmarks in the images;
orienting a noninvasive modulation apparatus relative to at least one of the landmarks; and
modulating the head of the caudate nucleus.

80. The method of claim 76, further comprising:
implanting a medical lead in communication with the portion of the patient's brain;
coupling the medical lead to a source of a modulating signal, wherein the source comprises one or more of an electrical pulse generator, or a reservoir of a therapeutic agent.

81. The method of claim 80, wherein the therapeutic agent is a pharmaceutical which interacts with one or more of:
signaling by glutamate, GABA, dopamine or serotonin;
neurotransmitter release;
ion channels having a normal physiological function of stabilizing or changing membrane potentials; or
second messenger systems.

82. The method of claim 80, wherein the therapeutic agent interacts with genetic mechanisms controlling or modulating with one or more of:
signaling by glutamate, GABA, dopamine or serotonin;
neurotransmitter release;
ion channels having a normal physiological function of stabilizing or changing membrane potentials; or
second messenger systems.

83. The method of claim 80, wherein the therapeutic agent administered via the medical lead modulates or otherwise interacts with one or more additional therapeutic agents.

84. The method of claim 83, wherein the one or more additional therapeutic agents are administered separately from the therapeutic agent.

85. The method of claim 84, wherein administration of the one or more therapeutic agents comprises injecting the one or more additional therapeutic agents into a fluid compartment, the fluid compartment comprising blood, or cerebrospinal fluid.

86. The method of claim 84, wherein administration of the one or more therapeutic agents comprises sequentially injecting the one or more additional therapeutic agents via the medical lead.

87. The method of claim 80, further comprising obtaining one or more indicators of conditions adjacent the medical lead, the one or more indicators comprising one or more of an electrical, a chemical or an electro-chemical stimulus-response assessment, an electrical recording, a sample of a physiological fluid which may contain chemical correlates of neuronal activity, or a biomarker of a disease process;
using the one or more indicators as a reference value;
applying the modulation via the medical lead;
obtaining one or more indicators of the environment adjacent the medical lead;
comparing the one or more indicators of the environment with the one or more indicators used as reference values; and
updating the modulating therapy.

88. The method of claim 87, wherein the updating comprises pausing the modulating or resuming the modulating.

89. The method of claim 76, wherein the phantom perception comprises tinnitus.

90. The method of claim 76, wherein the phantom perception comprises a voice.

91. The method of claim 76, wherein the modulating step comprises controlling the modulation by the patient.

92. The method of claim 76, further comprising suspending the modulating when the intrusiveness of the phantom is reduced to a level acceptable to the patient.

93. The method of claim 92, further comprising resuming the modulating when the intrusiveness of the phantom increases to a level unacceptable to the patient.

94. The method of claim 76, wherein the modulating step comprises stimulating the portion of the brain with a stimulus, the method further comprising steering the stimulus within the portion of the brain.

95. The method of claim 76, wherein the modulating step comprises bilaterally stimulating corresponding target sites in the brain.

96. The method of claim 76, wherein the modulating step comprises bilaterally stimulating complementary target sites in the brain.

97. The method of claim 76, wherein the modulating step comprises unilateral stimulation of the brain.

98. The method of claim 76, wherein the modulating step comprises forming a lesion in the brain.

99. The method of claim 76, wherein the modulating step comprises one of cooling, heating, or irradiating a portion of the brain.

100. A method of therapeutically treating localization of a phantom perception in a patient, said method comprising:
modulating a portion of the patient's brain, the portion comprising the substantia nigra pars reticulata (SNr); and
reducing localization or multisensory integration of the phantom by the patient.

101. The method of claim 100, wherein the portion of the patient's brain is modulated noninvasively.

102. The method of claim 100, further comprising repeating the modulating in subsequent sessions in order to continue relief from, or to restore relief from the localization or the integration of the phantom by the patient.

103. The method of claim 100, further comprising:
obtaining separate images of the brain, including the SNr, and bone or other radiodense or radiointense structures;
placing the images of the brain into a common coordinate system;
selecting landmarks in the images;
orienting a noninvasive modulation apparatus relative to at least one of the landmarks; and
modulating the SNr.

104. The method of claim 100, further comprising:
implanting a medical lead in communication with the portion of the patient's brain;
coupling the medical lead to a source of a modulating signal, wherein the source comprises one or more of an electrical pulse generator, or a reservoir of a therapeutic agent.

105. The method of claim 104, wherein the therapeutic agent is a pharmaceutical which interacts with one or more of:
signaling by glutamate, GABA, dopamine or serotonin;
neurotransmitter release;
ion channels having a normal physiological function of stabilizing or changing membrane potentials; or
second messenger systems.

106. The method of claim 104, wherein the therapeutic agent interacts with genetic mechanisms controlling or modulating with one or more of:
signaling by glutamate, GABA, dopamine or serotonin;
neurotransmitter release;
ion channels having a normal physiological function of stabilizing or changing membrane potentials; or
second messenger systems.

107. The method of claim 104, wherein the therapeutic agent administered via the medical lead modulates or otherwise interacts with one or more additional therapeutic agents.

108. The method of claim 107, wherein the one or more additional therapeutic agents are administered separately from the therapeutic agent.

109. The method of claim 108, wherein administration of the one or more therapeutic agents comprises injecting the one or more additional therapeutic agents into a fluid compartment, the fluid compartment comprising blood, or cerebrospinal fluid.

110. The method of claim 108, wherein administration of the one or more therapeutic agents comprises sequentially injecting the one or more additional therapeutic agents via the medical lead.

111. The method of claim 104, further comprising obtaining one or more indicators of conditions adjacent the medical lead, the one or more indicators comprising one or more of an electrical, a chemical or an electro-chemical stimulus-response assessment, an electrical recording, a sample of a physiological fluid which may contain chemical correlates of neuronal activity, or a biomarker of a disease process;
using the one or more indicators as a reference value;
applying the modulation via the medical lead;
obtaining one or more indicators of the environment adjacent the medical lead;
comparing the one or more indicators of the environment with the one or more indicators used as reference values; and
updating the modulating therapy.

112. The method of claim 111, wherein the updating comprises pausing the modulating or resuming the modulating.

113. The method of claim 100, wherein the phantom perception comprises tinnitus.

114. The method of claim 100, wherein the phantom perception comprises a voice.

115. The method of claim 100, wherein the modulating step comprises controlling the modulation by the patient.

116. The method of claim 100, further comprising suspending the modulating when the localization or the integration of the phantom is reduced to a level acceptable to the patient.

117. The method of claim 116, further comprising resuming the modulating when the localization or the integration of the phantom increases to a level unacceptable to the patient.

118. The method of claim 100, wherein the modulating step comprises stimulating the portion of the brain with a stimulus, the method further comprising steering the stimulus within the portion of the brain.

119. The method of claim 100, wherein the modulating step comprises bilaterally stimulating corresponding target sites in the brain.

120. The method of claim 100, wherein the modulating step comprises bilaterally stimulating complementary target sites in the brain.

121. The method of claim 100, wherein the modulating step comprises unilateral stimulation of the brain.

122. The method of claim 100, wherein the modulating step comprises forming a lesion in the brain.

123. The method of claim 100, wherein the modulating step comprises one of cooling, heating, or irradiating a portion of the brain.

124. A method of therapeutically treating reaction to a phantom perception in a patient, said method comprising:
modulating a portion of the patient's brain, the portion comprising the putamen; and
modifying the patient's responses to the phantom perception including gestures, messages or other sequential correlates of the phantom.

125. The method of claim 124, wherein the portion of the patient's brain is modulated noninvasively.

126. The method of claim 124, further comprising repeating the modulating in subsequent sessions in order to continue relief from, or to restore relief from the phantom perception.

127. The method of claim 124, further comprising:
obtaining separate images of the brain, including the putamen, and bone or other radiodense or radiointense structures;
placing the images of the brain into a common coordinate system;
selecting landmarks in the images;
orienting a noninvasive modulation apparatus relative to at least one of the landmarks; and
modulating the putamen.

128. The method of claim 124, further comprising:
implanting a medical lead in communication with the putamen;
coupling the medical lead to a source of a modulating signal, wherein the source comprises one or more of an electrical pulse generator, or a reservoir of a therapeutic agent.

129. The method of claim 128, wherein the therapeutic agent is a pharmaceutical which interacts with one or more of:
signaling by glutamate, GABA, dopamine or serotonin;
neurotransmitter release;
ion channels having a normal physiological function of stabilizing or changing membrane potentials; or
second messenger systems.

130. The method of claim 128, in which the therapeutic agent interacts with genetic mechanisms controlling or modulating with one or more of:
signaling by glutamate, GABA, dopamine or serotonin;
neurotransmitter release;
ion channels having a normal physiological function of stabilizing or changing membrane potentials; or
second messenger systems.

131. The method of claim 128, wherein the therapeutic agent administered via the medical lead modulates or otherwise interacts with one or more additional therapeutic agents.

132. The method of claim 131, wherein the one or more additional therapeutic agents are administered separately from the therapeutic agent.

133. The method of claim 132, wherein administration of the one or more therapeutic agents comprises injecting the one or more additional therapeutic agents into a fluid compartment, the fluid compartment comprising blood, or cerebrospinal fluid.

134. The method of claim 132, wherein administration of the one or more therapeutic agents comprises sequentially injecting the one or more additional therapeutic agents via the medical lead.

135. The method of claim 128, further comprising obtaining one or more indicators of conditions adjacent the medical lead, the one or more indicators comprising one or more of an electrical, a chemical or an electro-chemical stimulus-response assessment, an electrical recording, a sample of a physiological fluid which may contain chemical correlates of neuronal activity, or a biomarker of a disease process;
using the one or more indicators as a reference value;
applying the modulation via the medical lead;
obtaining one or more indicators of the environment adjacent the medical lead;
comparing the one or more indicators of the environment with the one or more indicators used as reference values; and
updating the modulating therapy.

136. The method of claim 135, wherein the updating comprises pausing the modulating or resuming the modulating.

137. The method of claim 124, wherein the phantom perception comprises tinnitus.

138. The method of claim 124, wherein the phantom perception comprises a voice.

139. The method of claim 124, wherein the modulating step comprises controlling the modulation by the patient.

140. The method of claim 124, further comprising suspending the modulating when the responses to the phantom are reduced to a level acceptable to the patient.

141. The method of claim 140, further comprising resuming the modulating when the responses to the phantom increase to a level unacceptable to the patient.

142. The method of claim 124, wherein the modulating step comprises stimulating the portion of the brain with a stimulus, the method further comprising steering the stimulus within the portion of the brain.

143. The method of claim 124, wherein the modulating step comprises bilaterally stimulating corresponding target sites in the brain.

144. The method of claim 124, wherein the modulating step comprises bilaterally stimulating complementary target sites in the brain.

145. The method of claim 124, wherein the modulating step comprises unilateral stimulation of the brain.

146. The method of claim 124, wherein the modulating step comprises forming a lesion in the brain.

147. The method of claim 124, wherein the modulating step comprises one of cooling, heating, or irradiating a portion of the brain.

148. A method of therapeutically treating identifiability of a phantom perception in a patient, said method comprising:
modulating a portion of the patient's brain, the portion comprising the caudate nucleus;
reducing or transforming sensory qualia of the phantom, or interfering with multisensory integration of the phantom; and
reducing the patient's ability to assign a specific identity to the phantom.

149. The method of claim 148, wherein the portion of the patient's brain is modulated noninvasively.

150. The method of claim 148, further comprising repeating the modulating in subsequent sessions in order to continue relief from, or to restore relief from the phantom perception.

151. The method of claim 148, further comprising:
obtaining separate images of the brain, including the caudate nucleus, and bone or other radiodense or radiointense structures;
placing the images of the brain into a common coordinate system;
selecting landmarks in the images;
orienting a noninvasive modulation apparatus relative to at least one of the landmarks; and
modulating the caudate nucleus.

152. The method of claim 148, further comprising:
implanting a medical lead in communication with the caudate nucleus;
coupling the medical lead to a source of a modulating signal, wherein the source comprises one or more of an electrical pulse generator, or a reservoir of a therapeutic agent.

153. The method of claim 152, wherein the therapeutic agent is a pharmaceutical which interacts with one or more of:
signaling by glutamate, GABA, dopamine or serotonin;
neurotransmitter release;
ion channels having a normal physiological function of stabilizing or changing membrane potentials; or
second messenger systems.

154. The method of claim 152, wherein the therapeutic agent interacts with genetic mechanisms controlling or modulating with one or more of:
signaling by glutamate, GABA, dopamine or serotonin;
neurotransmitter release;
ion channels having a normal physiological function of stabilizing or changing membrane potentials; or
second messenger systems.

155. The method of claim 152, wherein the therapeutic agent administered via the medical lead modulates or otherwise interacts with one or more additional therapeutic agents.

156. The method of claim 155, wherein the one or more additional therapeutic agents are administered separately from the therapeutic agent.

157. The method of claim 156, wherein administration of the one or more therapeutic agents comprises injecting the one or more additional therapeutic agents into a fluid compartment, the fluid compartment comprising blood, or cerebrospinal fluid.

158. The method of claim 156, wherein administration of the one or more therapeutic agents comprises sequentially injecting the one or more additional therapeutic agents via the medical lead.

159. The method of claim 152, further comprising obtaining one or more indicators of conditions adjacent the medical lead, the one or more indicators comprising one or more of an electrical, a chemical or an electro-chemical stimulus-response assessment, an electrical recording, a sample of a physiological fluid which may contain chemical correlates of neuronal activity, or a biomarker of a disease process;
using the one or more indicators as a reference value;
applying the modulation via the medical lead;
obtaining one or more indicators of the environment adjacent the medical lead;
comparing the one or more indicators of the environment with the one or more indicators used as reference values; and
updating the modulating therapy.

160. The method of claim 159, wherein the updating comprises pausing the modulating or resuming the modulating.

161. The method of claim 148, wherein the phantom perception comprises tinnitus.

162. The method of claim 148, wherein the phantom perception comprises a voice.

163. The method of claim 148, wherein the modulating step comprises controlling the modulation by the patient.

164. The method of claim 148, further comprising suspending the modulating when the ability to identify the phantom is reduced to a level acceptable to the patient.

165. The method of claim 165, further comprising resuming the modulating when the ability to identify the phantom increases to a level unacceptable to the patient.

166. The method of claim 148, wherein the modulating step comprises stimulating the portion of the brain with a stimulus, the method further comprising steering the stimulus within the portion of the brain.

167. The method of claim 148, wherein the modulating step comprises bilaterally stimulating corresponding target sites in the brain.

168. The method of claim 148, wherein the modulating step comprises bilaterally stimulating complementary target sites in the brain.

169. The method of claim 148, wherein the modulating step comprises unilateral stimulation of the brain.

170. The method of claim 148, wherein the modulating step comprises forming a lesion in the brain.

171. The method of claim 148, wherein the modulating step comprises one of cooling, heating, or irradiating a portion of the brain.
